(19) United States
(12) Patent Application Publication
(10) Pub. No.: US 2020/0239597 A1
(43) Pub. Date: Jul. 30, 2020

(54) AN ANTI-APOPTOTIC FUNCTION OF PKM2 AND INTRACELLULARLY EXPRESSED SCFV ANTIBODIES

(71) Applicants: Donald NEWMEYER, La Jolla, CA (US); Tong LIU, La Jolla, CA (US); La Jolla Institute for Allergy and Immunology, La Jolla, CA (US)

(72) Inventors: Donald Newmeyer, San Diego, CA (US); Tong Liu, San Diego, CA (US)

(21) Appl. No.: 16/638,099

(22) PCT Filed: Aug. 9, 2018
(86) PCT No.: PCT/US18/46142

§ 371 Date: Feb. 10, 2020

Related U.S. Application Data
(60) Provisional application No. 62/543,264, filed on Aug. 9, 2017.

(51) Int. Cl. C07K 16/40; A61K 39/395; A61K 38/45; A61K 47/68; A61K 9/127

(52) U.S. Cl. CPC C07K 16/40; A61K 39/3955; A61K 38/45; C12Y 207/0104; C07K 2317/82; A61K 9/127; C07K 2317/565; C07K 2317/24; C07K 2317/622; A61K 47/6811

(57) ABSTRACT

This application generally relates to the field of methods, systems and compositions for addressing diseases associated with apoptotic cell death, including autoimmune diseases and inflammatory diseases, and more particularly to such methods, systems and compositions that use antibodies having binding specificity to PKM2.

### CROSS-REFERENCE TO RELATED PATENT APPLICATION

This application claims the benefit under 35 U.S.C. 119(e) to U.S. Provisional Application No. 62/543,264 filed Aug. 9, 2017, the contents of which are hereby incorporated by reference in its entirety.

### STATEMENT REGARDING FEDERALLY SPONSORED RESEARCH OR DEVELOPMENT

This invention was made with government support under grant numbers R01 GM62289 and R01 CA179087, awarded by the National Institute of Health (NIH). The U.S. Government has certain rights in the invention

### TECHNICAL FIELD

This application generally relates to the field of methods, systems and compositions for addressing diseases associated with apoptotic cell death, including autoimmune diseases and inflammatory diseases, and more particularly to such methods, systems and compositions that use antibodies having binding specificity to PKM2.

### SEQUENCE LISTING

In accordance with 37 CFR 1.52(e)(5), the present specification makes reference to a Sequence Listing (submitted electronically as a .txt file named “SeqListing.txt” on Aug. 9, 2018). The .txt file was generated on Aug. 8, 2018 and is 8 kb in size. The entire contents of the Sequence Listing are herein incorporated by reference.

### BACKGROUND

Apoptosis is a cellular suicide process that is important for certain aspects of normal animal development (Tuzlak et al., 2016) and is dysregulated in various diseases, especially cancer (e.g. Brown and Attardi, 2005; Elmore, 2007). Members of the Bcl-2 protein family act at the mitochondrial outer membrane to regulate the central events in apoptotic cell death (Bender and Martinou, 2013; Czabotar et al., 2014; Gillies and Kuwana, 2014; Kluck et al., 1997; Kuwana et al., 2002; Li and Dewson, 2015; Lopez and Tait, 2015; Newmeyer et al., 1994; Newmeyer and Ferguson-Miller, 2003; Volkmann et al., 2014). Venetoclax, a drug targeting Bcl-2, is currently approved for the treatment of a refractory form of CLL (Croce and Reed, 2016; Green, 2016), and other drugs that directly target Bcl-2-family proteins are now in cancer clinical trials (Brown et al., 2015; Debrincat et al., 2015; Gandhi et al., 2011; Johnson-Farley et al., 2015; Kipps et al., 2015; Leverson et al., 2015; Lieber et al., 2015; Roberts et al., 2015; Sarosiek and Letai, 2016; Swiecicki et al., 2016).

Bcl-2-family proteins function in a complex network of heterodimeric interactions that collectively decide between cell survival and death (Volkmann et al., 2014). Several Bcl-2 subfamilies carry out different functions (Chipuk et al., 2010). In particular, the proteins Bax and Bak comprise the effector subfamily responsible for the critical mitochondrial events in cell death. Genetic and in vitro studies (Cheng et al., 2001; Du et al., 2011; Kuwana et al., 2005a; Kuwana et al., 2002; Walensky et al., 2006) have shown that Bax/Bak can be activated by transient interactions with other Bcl-2 family proteins belonging to the “BH3-only” category (including Bim, Bid, Puma, and others.) Once activated, Bax/Bak undergo conformational changes to become fully integrated in the MOM. As a result, these proteins form large, heterogeneous membrane pores (Gillies et al., 2015; Schafer et al., 2009), in an event known as mitochondrial outer membrane permeabilization (MOMP) (Bender and Martinou, 2013; Chipuk and Green, 2008; Youle and Strasser, 2008). MOMP allows soluble mitochondrial proteins (e.g., cytochrome c, Smac and Omi) to escape into the cytoplasm, where they trigger the activation of caspase proteases that carry out the cell death program.

MOMP, and in turn cell death, is largely governed by this complex interplay among Bcl-2-family proteins (Chen et al., 2005; Kuwana et al., 2005b; Kuwana et al., 2002; Llambi et al., 2011). The importance of MOMP for cancer therapy is underscored by the finding that the in vitro response of mitochondria from patient tumor samples to BH3 domain peptides can often predict the effect of therapy (Del Gaizo Moore and Letai, 2013; Montero et al., 2015; Suryani et al., 2014).

Bcl-2 family members can also be regulated by proteins outside the Bcl-2 family. For example, p53 can act at mitochondria both to activate Bax directly and to sequester Bcl-xL (Chipuk et al., 2004). Similarly, the Retinoblastoma protein pRB is reported to translocate to mitochondria to promote Bax activation in a non-transcriptional manner (Hilgendorf et al., 2013), and oncogenes such as Myc and Ras also modulate the expression of key Bcl-2-family proteins (Juin et al., 2013). The ability of proto-oncoproteins to inhibit or activate apoptosis can be seen as an important facet of their homeostatic function, inasmuch as cell death serves as a critical counterbalance to cell proliferation.

In view of the foregoing, the present disclosure proposes methods, systems and compositions for addressing diseases associated with apoptotic cell death.

### SUMMARY

This Summary is provided to introduce a selection of concepts in a simplified form that are further described below in the Detailed Description. This Summary is not intended to identify key aspects or essential aspects of the claimed subject matter.

As embodied and broadly described herein, the present disclosure relates to a method for prevention or treatment of a disease promoting apoptotic cell death in a subject, comprising: contacting, in a cell of the subject, an antibody or fragment thereof with a pyruvate kinase M2 (PKM2) protein, the antibody or fragment thereof having binding specificity to PKM2.

As embodied and broadly described herein, the present disclosure also relates to a method for prevention or treatment of apoptotic cell death in a subject, the apoptotic cell death being associated with mitochondrial outer membrane permeabilization (MOMP), the method comprising: contacting, in a cell of the subject, an antibody or fragment thereof with a pyruvate kinase M2 (PKM2) protein, the antibody or fragment thereof having binding specificity to PKM2.

As embodied and broadly described herein, the present disclosure also relates to a single chain (scFv) antibody comprising the amino acid sequence set forth in SEQ ID NO: 1.

As embodied and broadly described herein, the present disclosure also relates to a single chain (scFv) antibody comprising the amino acid sequence set forth in SEQ ID NO: 2.

As embodied and broadly described herein, the present disclosure also relates to an antibody having binding specificity to pyruvate kinase M2 (PKM2), the antibody comprising the CDR1(H) set forth in SEQ ID NO: 3, the CDR2(H) set forth in SEQ ID NO: 4 and the CDR3(H) set forth in SEQ ID NO: 5; or the CDR1(H) set forth in SEQ ID NO: 9, the CDR2(H) set forth in SEQ ID NO: 10 and the CDR3(H) set forth in SEQ ID NO: 11, wherein the CDR1(H), CDR2(H) and CDR3(H) are linked in tandem.

As embodied and broadly described herein, the present disclosure also relates to a humanized, single chain (scFv) or chimeric antibody having binding specificity to a pyruvate kinase M2 (PKM2) epitope comprising at least a portion of the amino acid sequence set forth in SEQ ID NO: 15.

As embodied and broadly described herein, the present disclosure also relates to a humanized, single chain (scFv) or chimeric antibody, the antibody or fragment thereof having binding specificity to a PKIVI2 conformational epitope including amino acid residues contained in the amino acid sequence set forth in SEQ ID NO: 15.

All features of exemplary embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with the accompanying Figures.

### BRIEF DESCRIPTION OF THE DRAWINGS

A detailed description of specific exemplary embodiments is provided herein below with reference to the accompanying drawings in which:

FIG. 1A is a non-limiting histogram representation of an assay for selection of intrabodies that rescue cells from BimS-induced apoptosis. There is shown the result of enrichment in two rounds of selection. For the first round: 293T cells were first infected with a lentiviral human naïve scFv library. Then, 1×10⁵ of the scFv-expressing cells were transiently transfected with BimS (using either 4 μg/ml or 6 μg/ml of plasmid DNA input per reaction) under the control of the EF-1αpromoter, as indicated in Methods. Lentiviral DNA was recovered from these rescued cells and used for a second round of selection. In the second round, many more cells (about 40%) were rescued from BimS-induced apoptosis.

FIG. 1B is a non-limiting graph representation that shows the results of individual DNA sequences isolated from FIG. 1A that were expressed in 293T cells for testing of their ability to protect cells from apoptosis induced by transfection with BimS. After three rounds of selection, intrabody coding sequences were amplified by PCR and subcloned into a plasmid for expression in E. coli. Individual DNA sequences were sequenced and expressed in 293T cells for testing of their ability to protect cells from apoptosis induced by transfection with BimS. The percentage of viable cells, relative to cells not transfected with BimS, was assayed.

FIG. 1 C is a photograph of a non-limiting SDS-PAGE gel with silver stain from an immunoprecipitation assay using antibodies encoded by DNA sequences isolated from FIG. 1B. These results show that some intrabodies arising from the selection procedure immunoprecipitate specific cellular proteins. Intrabodies that rescued cells from BimS-induced death were chosen for pull-down analysis as described in Methods. Left panel: Triton™-X-100 (1%) cell extracts were incubated with anti-FLAG beads, then proteins eluted with 3×FLAG peptide and separated by SDS-PAGE with silver stain. Specific bands are marked with dots; some bands (e.g. at 37 and 70 kD) are nonspecific. Clones 5, 7 and 12 (independent isolates) pulled down a 55-kD protein now identified as pyruvate kinase M2, while clone 19 pulls down several specific bands (not studied here). The bands near 25 kD are the scFv polypeptides, whose expression levels varied.

FIG. 1D is a photograph of a non-limiting immunoprecipitation-western of lysates from cells expressing the IB5 clone. The lysates were incubated with anti-FLAG beads, and coprecipitating proteins were eluted with FLAG₂ peptide (lane 3). Immunoblots were probed with antibody to PKM2 (left) or PKM1 (right). Purified PKM2 (lane 1) and PKM1 (lane 2) were controls for antibody specificity.

FIG. 1E is a non-limiting sequence alignment comparison between the amino acid sequence of the IB5 clone and the IB12 clone. Protein sequences of IB5 and IB12 are dissimilar, underscoring the functional importance of their common target, PKM2. Red boxes: heavy chain Complementarity Determining Regions (CDRs); green boxes: light chain CDRs; magenta type: FLAG tag. The selected intrabody plasmids were sequenced by Sanger sequencing. Sequences were analyzed with Vbase2.

FIG. 2A is a photograph of a non-limiting assay in petri dishes to assess whether IB5 produces clonogenic survival, despite BimS expression. Control or IB5-expressing cells were transfected with BimS cDNA, and after 5 d, the plates were fixed with 6.0% glutaraldehyde and stained with 0.5% crystal violet. Top: example crystal violet-stained plate; bottom: average colony counts from three independent experiments, ±SEM.

FIG. 2B is a non-limiting graph representation of a transfection assay to assess the effect of IB5 in cells transfected with pro-apoptotic proteins BimS and tBid encoding cDNA. There it is shown that IB5-expressing 293T cells were protected from death induced by transient expression of tBid or BimS. 293T cells were transfected treated with 1 g/ml tBid or BimS cDNA. Surviving cells were counted after 72 h.

FIG. 2C shows a photograph of a non-limiting assay in petri dishes (left panel) and a nonlimiting graph representation (right panel) of an assay to assess whether IB5 expression rescues U2OS and HCT116 cells clonogenically from BimS-induced death. Left: examples of crystal violetstained plates; right: as cells did not typically grow as discrete colonies, the present inventors measured colony area, as a percentage of total plate area. The corresponding graph is shown on the right panel.

FIG. 3A is a non-limiting graph representation that shows the results of genetic deletion of the M2 isoform of pyruvate kinase over the protective effect of scFv #5, alone or in combination with TEPP-46, over cell death induced by transfection of BimS expression plasmid. Wild type (WT) mouse embryonic fibroblasts (MEFs), PKM2-deficient MEFs, or PKM2-deficient MEFs reconstituted with WT or mutant PKIM2 cDNA, were infected or not with IB5, then later transfected with BimS expression plasmid. Surviving cells were counted 48 h afterwards. Note that only WT cells or PKM2-deficient MEFs reconstituted with WT PKM2 exhibited cytoprotective activity of IB5.

FIG. 3B shows a photograph of a non-limiting assay in petri dishes (left panel) and a nonlimiting graph representation (right panel) of an assay to assess whether IB5-induced clonogenic rescue of 293T cells from BimS was enhanced by treatment with TEPP-46. Control 293T or IB5-expressing cells were incubated with or without 27 g/ml TEPP-46 for 3 h, then transfected with BimS cDNA in a further 24-h incubation also including TEPP-46 or vehicle. The plates were fixed and stained with crystal violet after 1 week. The total area of colonies (a measure of the total mass of proliferating cells) formed in each well from were quantified using ImageJ software; mean±SEM are shown from 3 independent experiments.

FIG. 3C is a non-limiting graph representation that shows the results of genetic mutation (K422R) of the M2 isoform of pyruvate kinase over the protective effect of scFv #5 over cell death induced by transfection of BimS expression plasmid.

FIG. 4A shows a photograph of a non-limiting blue native gel electrophoresis of E. coli cell extract expressing a monovalent form of scFv 5 incubated with WT PKM2 or mutant PKM2 (K422R) (left panel), or with PKM1, WT PKM2 or mutant PKM2 (K422R) (right panel). Left: A monovalent form of scFv 5, produced in E. coli, induced tetramer formation in WT PKM2 along with a bandshift whose magnitude was dependent on the input amount of scFv 5. The mutant PKM2 (K422R) was constitutively tetrameric and did not exhibit a bandshift in the presence of scFv 5. Reaction volume was 20 μl. Right: scFv 5 did not produce a band shift with recombinant PKM1, which ran as a tetramer; added FBP produced the tetramer form of WT PKM2 (lanes 6-8).

FIG. 4B shows a non-limiting graph representation that shows the results of an assay assessing whether scFv 5 stimulated glycolytic activity of WT PKM2 or mutants thereof. scFv 5 stimulated glycolytic activity of WT PKM2. Activity was measured by Kinase-Glo® Plus Luminescent Kinase Assay kit (promega), using ADP and PEP as substrates), with PKM2 at 50 nM. Shown are values with the basal activity of PKM2 alone subtracted out. Inset: Stimulation of PKM2 activity by the allosteric activator fructose 1,6-bisphosphate (FBP).

FIG. 4C shows a non-limiting graph representation that shows the results of an assay assessing glycolytic activity (via luminescence) of WT PKM2 or PKM2 (K422R) in presence or absence of IB5, FBP or TEPP, as shown.

FIG. 5A shows a photograph of a non-limiting assay in petri dishes assessing the cytoprotective effect (after 7 passages) of IB5 on PKM2-deficient MEFs reconstituted with WT or mutant PKM2 cDNA, which were transfected with BimS expression plasmid. The plates were fixed and stained with crystal violet after 1 week and the total area of colonies were counted as above. The SD and P value was calculated from 6 individual plates.

FIG. 5B shows a non-limiting graph representation that shows the results of an assay that assesses the assessing the cytoprotective effect (after 4 or 7 passages) of IB5 on PKM2-deficient MEFs reconstituted with WT or mutant PKM2 cDNA, which were transfected with BimS expression plasmid. Quantification of clonogenic survival for passages 4 and 7 shows that at both early and later passages, the K422R mutant supported the cytoprotective effect of IB5; at later passage, this mutant protected cells to a substantial degree even in the absence of IB5 expression.

FIG. 6A shows a photograph of a non-limiting immunoblot of a cytochrome c release assay in control (top—left panel) or IB5 intrabody-expressing (bottom-left panel) 293T cells. Control (top) or Intrabody-expressing (bottom) 293T cells were collected and the mitochondrial fraction was isolated by differential centrifugation. To induce MOMP, recombinant cBid protein was added at the indicated concentrations. After incubation for 30 min at 37° C., samples were centrifuged, and cytochrome c (cyt c) content in mitochondrial pellet fractions was analyzed by immunoblot. A representative of three independent experiments is shown. Right panel: densitometric quantification of average cytochrome c content ±SEM from three independent experiments.

FIG. 6B shows non-limiting photographs of immunoblotting of several Bcl-2 family proteins expression levels in cells following IB5 expression or incubation with TEPP-46, or both. Levels of several Bcl-2 family proteins were unchanged following IB5 expression or incubation with TEPP-46, or both. Cell lysates from 293T cells infected with and without IB5 and incubated with and without TEPP-46 (27 μM) were separated on SDS-12% polyacrylamide gels. Bcl-2 family proteins were detected by immunoblotting. The bands were quantified using ImageJ and normalized to the control cell lysate on the leftmost lane.

FIG. 7A shows non-limiting images of mitochondria visualized by confocal fluorescence microscopy after staining with Tom20 antibodies in PKM2-deficient MEFs reconstituted with WT PKIIM2 or PKM2(K422R) cDNA, which were infected or not with IB5 lentivirus. IB5 expression with WT PKM2 increased mitochondrial length, and PKM2 (K422R) expression increased mitochondrial length even in the absence of IB5. PKM2-deficient MEFs reconstituted with WT PKIM2 or PKM2(K422R) cDNA, were infected or not with IB5 lentivirus. IB5 expression with WT PKIM2 increased mitochondrial length, and PKM2 (K422R) expression increased mitochondrial length even in the absence of IB5. Mitochondria were visualized by fluorescence microscopy after staining with Tom20 antibodies. Representative confocal images are shown.

FIG. 7B shows a non-limiting graph representation that shows the results of an assay measuring mitochondrial length scores of cells as in FIG. 7A. Cells were analyzed 3 d after transfection with the indicated cDNA constructs (mean±s.e.m. of 3-5 experiments of 120-200 random selected cells.

FIG. 7C shows non-limiting photographs of immunoblotting of Mfn1 protein levels in MEFs reconstituted with PKM2 WT and K422R mutant. IB5 expression upregulated Mfn1 protein in MEFs reconstituted with PKM2 WT and K422R mutant.

FIG. 7D shows a photograph of a non-limiting assay in petri dishes assessing the IB5 cytoprotective effect over BimS-induced death in WT MEFs, Mfn2-null MEFs and Mfn1-null MEFs. IB5 expression rescued WT and Mfn2-null MEFs from BimS-induced death but failed to rescue Mfn1-null MEFs. WT, Mfn1- or Mfn2-deficient MEFs were infected or not with IB5 lentivirus, then 1×10⁵ cells were plated and transfected with BimS expression plasmid. The plates were fixed and stained with crystal violet after 1 week and the total areas of colonies were measured as above. Mean, SD and P values were calculated from 5 individual plates.

FIG. 8A shows a non-limiting graph representation that shows the results of an assay assessing the cytoprotective effect of IB5 over BimS-induced death in PKIM2 siRNA ablated cells or control NF-kB p50-specific siRNA ablated cells. siRNA knockdown of PKM2 ablated the protective effect of IB5 in 293T cells. 5×10⁵ cells were incubated per well for 12 h, then cells were either mock-transfected, transfected with 30 nM PKM2-specific siRNA (si M2), or transfected with NF-kB p50-specific siRNA (si p50). After a further 36-h incubation, samples of the same siRNAs were added along with 4 μg of BimS cDNA in fresh medium. Viable cells were counted after another 48-h incubation.

FIG. 8B shows that expression of IB5 had no effect on expression of endogenous PKM2 or Bim EL and L isoforms.

FIG. 9 shows a photograph of a non-limiting assay in petri dishes assessing the IB5 cytoprotective effect over BimS-induced death in breast cancer-derived cell lines MDA-MB231 (left-bottom panel) and lung metastatic derivative, MDA-MB231-LM2 (left-top panel). The right panel shows a non-limiting graph representation of the results obtained in the left top and left bottom panels. Control or IB5-expressing cells were transfected with BimS cDNA. The plates were fixed and stained with crystal violet after 12 days and the total areas of colonies were measured. Mean, SD and P values were calculated from 3 individual plates.

FIG. 10 shows a photograph of a non-limiting assay in petri dishes assessing the IB5 cytoprotective effect over BimS-induced death in HCT116 and U2OS cells, in presence of 150 nM etoposide or 1 μM Staurosporine. 5×10⁵ HCT116 and U2OS cells were plated and transfected with BimS expression plasmid including 150 nM etoposide or 1 μM Staurosporine. The plates were fixed and stained with crystal violet after 5 days and the total areas of colonies were measured. Mean, SD and P values were calculated from 3 individual plates.

FIG. 11 shows a non-limiting western blot against Mfn1 (top panel), Mfn2 (bottom panel) and actin in wild type or Mfn1-Mfn2 null mutants, as shown.

FIG. 12 shows a non-limiting sequence alignment representation between the amino acid sequence encoded by exon 9 and by exon 10 of the PMK gene.

In the drawings, exemplary embodiments are illustrated by way of example. It is to be expressly understood that the description and drawings are only for the purpose of illustrating certain embodiments and are an aid for understanding. They are not intended to be a definition of the limits of the invention.

### DETAILED DESCRIPTION

A detailed description of one or more embodiments of the invention is provided below along with accompanying figures that illustrate the principles of the invention. The invention is described in connection with such embodiments, but the invention is not limited to any embodiment. The scope of the invention is limited only by the claims. Numerous specific details are set forth in the following description in order to provide a thorough understanding of the invention. These details are provided for the purpose of non-limiting examples and the invention may be practiced according to the claims without some or all of these specific details. For the purpose of clarity, technical material that is known in the technical fields related to the invention has not been described in detail so that the invention is not unnecessarily obscured.

PKIM2 can inhibit the central mechanism of mitochondrial apoptosis

To discover molecules regulating the core mechanism of mitochondria-dependent cell death, the present inventors developed an unbiased functional selection approach that used libraries of “intrabodies”: intracellularly expressed single-chain antibodies (scFv). The inventors found that some of the selected intrabodies specifically recognized a key metabolic regulatory protein, pyruvate kinase M2 (PKM2). This indicates that PKM2, aside from its well-documented role in glycolytic metabolism, could also have an expressly anti-apoptotic function.

PKM2 is an important regulator of tissue homeostasis, as well as tumor growth and metabolism (e.g. Christofk et al., 2008b) and is currently a subject of intense research (reviewed in Cantor and Sabatini, 2012; Iqbal et al., 2014a; Li et al., 2014; Wong et al., 2015). PKM2 is a glycolytic enzyme that promotes the “Warburg effect”, also termed aerobic glycolysis, in which cells exhibit increased glucose to lactate conversion even in the presence of oxygen (Hitosugi et al., 2009). In cancer cells, PKM2 is typically expressed preferentially over its related isoform, PKM1, even when the tissue of origin does not express PKM2. Hypothetically, cancers gain some selective advantage from the highly regulated functions of PKM2. The adaptive metabolic functions of PKM2 also come into play in some cell types that quickly transition to a proliferative state, such as LPS-activated macrophages (Palsson-McDermott et al., 2015).

PKM1 and PKM2 are generated from transcripts of the PKM gene by alternative mRNA splicing. Both isoforms can catalyze the last step in glycolysis, in which phosphoenolpyruvate (PEP) and ADP are converted to pyruvate and ATP. Isoforms M1 and M2 are identical except for the region encoded by the one alternatively spliced exon (exon 9 for PKM1 and 10 for PKM2), yielding a difference in only 22 amino acids. PKM1 exists as a constitutively active tetramer, whereas PKM2 is subject to many forms of regulation. Various metabolites, including fructose-1,6-bisphosphate (FBP), serine, phenylalanine, and triiodo-L-thyronine (T3), can allosterically regulate PKM2's glycolytic activity (Hitosugi et al., 2009; Morgan et al., 2013). In vitro biochemical studies have shown that PKM2 exists in equilibrium between a glycolytically active tetramer form and less active dimer or monomer forms (Gui et al., 2013; Mazurek, 2011). Based on crystallographic data, it has also been proposed that PKM2 tetramers can transition between inactive T-state and active R-state conformations (Wang et al., 2015a).

### Glycolytic and Nonglycolytic Functions of PKIM2

Paradoxically, it is the ability of PKM2's glycolytic activity to be reduced that favors rapid cell proliferation. Reduced PK activity correlates with increased biosynthesis of metabolites important for cell proliferation, potentially explaining why tumor cells prefer the M2 isoform (Christofk et al., 2008a; Hitosugi et al., 2009). Consistent with this idea, treatment of cells with small-molecule activators of PKM2 (Anastasiou et al., 2012; Parnell et al., 2013) or the replacement of PKM2 with the constitutively active isoform, PIKM1 (Christofk et al., 2008b), can reduce cell proliferation in some situations. In primary MEFs, deletion of PKM2 results in increased PIKM1 expression, and this in turn impairs nucleotide availability for DNA synthesis, thereby inhibiting cell cycle progression (Lunt et al., 2015).

PKVI2 is reported also to have nonglycolytic functions. Many PKM2 interaction partners have been described, including multiple transcription factors (Wu and Le, 2013). For example, PKM2 is reported to cooperate with Hif-la to regulate the transcription of multiple glycolysisrelated proteins, which contribute to metabolic remodeling and the Warburg effect (Luo et al., 2011; Luo and Semenza, 2011; Palsson-McDermott et al., 2015; Palsson-McDermott and O'Neill, 2013). These transcriptional functions require the nuclear import of PKM2 (Gao et al., 2012; Luo et al., 2011; Luo and Semenza, 2011; Lv et al., 2013). PKM2's nuclear translocation can be promoted by EGFR activation (Yang et al., 2011) and regulated by Erkl/2 and JMJD5 (Wang et al., 2014; Yang et al., 2012b). In the nucleus, PKIM2 can promote 0-catenin transactivation, leading to the expression of cyclin D1 and tumorigenesis (Yang et al., 2011). A PKIM2-activating compound, TEPP-46, which causes PKM2 tetramerization, inhibits Hif-la-dependent transcriptional effects (Palsson-McDermott et al., 2015), supporting the idea that the dimeric form of PKM2 is responsible for transcriptional functions. Dimeric PKM2 is also reported to possess protein kinase activity, targeting multiple oncogenic factors (Gao et al., 2012; Jiang et al., 2014a; Jiang et al., 2014b; Yang et al., 2012a). However, PKM2 protein kinase activity is controversial, as Vander Heiden and colleagues found no evidence of protein kinase activity for PKM2 in cell lysates (Hosios et al., 2015).

How PKM2 could Regulate Apoptosis is Unclear

In some cases, PKIM2 ablation can produce or enhance cell death (Chu et al., 2015; Gines et al., 2015; Kim et al., 2015b; Li et al., 2016; Shi et al., 2010; Wang et al., 2015b; Yuan et al., 2016; Zhou et al., 2014). Precisely how PKM2 affects apoptosis is unclear. PKM2 silencing has been reported to stabilize proapoptotic Bim (Hu et al., 2015) or downregulate the expression of the anti-apoptotic proteins Bcl-xL or Mcl-1 (Dong et al., 2015; Kwon et al., 2012). However, PKM2 knockdown produces an artificial situation. PKM2 has multiple functions that may be regulated independently, and experiments in which this protein is ablated would involve a simultaneous loss of all these activities, along with a compensatory upregulation of PKM1, making interpretation difficult. In contrast to the studies just mentioned, Sabatini and colleagues showed that the inhibition of PKM2 activity under ischemic conditions had the effect of promoting cell survival, rather than cell death (Kim et al., 2015a). The cells bordering necrotic foci in gliomas expressed higher levels of the enzyme SHMT2, leading to an allosteric inhibition of PKM2's glycolytic activity. This provided a significant protection from ischemic cell death. In another ischemia model, these authors found that overexpression of PKM2 or treatment with the PKM2-activating compound TEPP-46 eliminated the increased cell viability produced by SHMT2. It is unclear whether this connection between reduced PKM2 activity and survival is a general phenomenon, or only applies to certain cancer cell subsets or environments.

In contrast to studies emphasizing PKM2 loss of function, the present inventors' results now show that PKM2 possesses a positive cytoprotective function that can be activated by a PKM2-specific intracellularly expressed single-chain antibody (intrabody). The present inventors show that this latent function of PKM2 counteracts the central Bax/Bak-dependent mitochondrial apoptotic mechanism. IB5 produced a cytoprotective effect in conjunction with a stably tetrameric mutant PKM2 (K422R), arguing that the anti-apoptotic effect involves the cytoplasmic tetramer form of PKM2. The K422R mutant also produced BimS-resistance in MEFs at late passages, even in the absence of IB5 expression. This mutant's ability to counteract the central apoptotic pathway could provide a selective advantage for these cells, and indeed this mutation was spontaneously selected in Bloom syndrome patient tumor cells. The IB5/PKM2-induced cytoprotective function depended in part on upregulation of the mitochondrial fusion-related protein Mitofusin-1 (Mfn1). Without being bound by any theory, the present inventors propose that PKM2 can activate an Mfn1-dependent general anti-apoptotic pathway, which could help explain why human cancer cells often preferentially express the M2 isoform of pyruvate kinase.

### Sequences

The present disclosure makes reference to various sequences which are set forth in the accompanying sequence listing. These sequences are reproduced below:

SEQ ID NO: 1-amino acid sequence of the intrabody
clone IB12:
MAQVQLVQSGGGLVKPGGSLRLSCTASGFTFSTYWMHWFRQAPGKGLLWV
SRINPDGSATIYADSVKGRFTISRDNAKNSLYLQMNSLRDEDTAVYYCAR
GHPLSGYPGYFDYWGQGTLVTVSSGGGGSGGGGSGVADPRLCSLSRLPSL
HLLEHQQSHLHFTSGINVGAYRIYWYQQKPGSPPQFLLRYKSDSDKQQGS
GVPSRFSGSRDASANAGILLISGLRSEDEADYYCAIWHSSAWVFGGGTKL
TVLWGSGLASVDYKDDDDK.
SEQ ID NO: 2-amino acid sequence of the intrabody
IB5:
MAQVQLVETGPGLVKPSETLSLRCTVSGGSFDNYYWNWIRQPPGKGLEYI
GYVFPSTGATNYNPSLGSRVTISLDTSKNQFSLTLTSVTTADTAIYYCVR
SGHDLWTGSTWFDPWGQWTTVTVSSGGGGSGGGGSGGGGSEIVLTQSPGT
LSLSPGERATLSCRASQSVSSSYLAWYQQKPGQAPRLLIYGASSRATGIP
DRFSGSGSGTDFTLTISRLEPEDIAVYYCQQRSNWPRTFGQGTKVEIKRG
LGGLASVDYKDDDDK.
SEQ ID NO: 3; predicted amino acid sequence of the
CDR1(H) for IB5:
GSFDNYYW.
SEQ ID NO: 4; predicted amino acid sequence of the
CDR2(H) for IB5:
FPSTGATN.
SEQ ID NO: 5; predicted amino acid sequence of the
CDR3(H) for IB5:
HDLWTGSTWF.
SEQ ID NO: 6-predicted amino acid sequence of the
CDR1(L) for IB5:
SQSVSSSYLA.
SEQ ID NO: 7-predicted amino acid sequence of the
CDR2(L) for IB5:
ASSRAT.
SEQ ID NO: 8-predicted amino acid sequence of the
CDR3(L) for IB5:
QRSNWPRT.
SEQ ID NO: 9-predicted amino acid sequence of the
CDR1(H) for IB12:
FTFSTYWM.
SEQ ID NO: 10-predicted amino acid sequence of the
CDR2(H) for IB12:
NPDGSATI.
SEQ ID NO: 11-predicted amino acid sequence of the
CDR3(H) for IB12:
HPLSGYPGYF.
SEQ ID NO: 12-predicted amino acid sequence of the
CDR1(L) for IB12:
GINVGAYRIY.
SEQ ID NO: 13-predicted amino acid sequence of the
CDR2(L) for IB12:
SDSDKQ.
SEQ ID NO: 14-predicted amino acid sequence of the
CDR3(L) for IB12:
IWHSSAWV.
SEQ ID NO: 15-amino acid sequence encoded by exon
10 of the PKM gene (present in PKM2):
IAREAEAAIYHLQLFEELRRLAPITSDPTEATAVGAVEASFKCCSGAIIV
LTKSG.
SEQ ID NO: 16-amino acid sequence encoded by exon
9 of the PKM gene (present in PKM1):
IAREAEAAMFHRKLFEELVRASSHSDTDLMEAMAMGSVEASYKCLAAALI
VLTESG.

### Definitions

Unless otherwise defined, all technical and scientific terms used herein have the same meaning as commonly understood by a person of ordinary skill in the art to which the present invention pertains. As used herein, and unless stated otherwise or required otherwise by context, each of the following terms shall have the definition set forth below.

The term “modified” used with respect to the antibody of the present disclosure refers to a substance that binds to the antibody directly or indirectly. Examples of such substance include peptides, lipids, saccharides, and naturally occurring or synthetic polymers, but are not limited thereto.

The present disclosure makes reference to a method which includes a step of causing the intracellular expression of an antibody in a cell having the PIKM2 protein.

In one embodiment, the intracellular expression of the antibody may be obtained by introducing the antibody into the cell by administration to the subject, of the antibody.

In one practical implementation, the antibody may be modified to include another substance. The antibody may have any modification as long as the activity of binding to its epitope is maintained. In addition, the antibody may be imparted additional function by the modification. Examples of the additional function include target-directing property, stability, and cell membrane permeability, but are not limited thereto.

For example, the modification may include introduction of a cell membrane permeable substance. The intracellular structure is commonly shielded from the external environment by a cell membrane. Thus, it is difficult to effectively introduce an extracellular substance into a cell. However, certain substances have cell membrane permeability, and can be introduced into a cell without being blocked by a cell membrane. A substance not having cell membrane permeability can be imparted the cell membrane permeability by being modified with such a substance having cell membrane permeability (cell membrane permeable substance).

Thus, the antibody of the present disclosure can be modified with a cell membrane permeable substance so as to be effectively introduced into a cell. Furthermore, herein, “cell membrane permeability” refers to a property of permeating a cell membrane of a mammal to enter the cytoplasm. In addition, “cell membrane permeable substance” refers to a substance having the “cell membrane permeability”.

Examples of the cell membrane permeable substance include membrane fusion liposomes, and cell membrane permeable peptides, but are not limited thereto.

For example, the membrane fusion liposome is fused with a cell membrane, whereby to release the contents into the cell. The membrane fusion liposome can be prepared, for example, by modifying the liposome surface with a substance having membrane fusion property. Examples of the membrane fusion liposome include pH-sensitive liposome (Yuba E, et at, J. Control. Release, 149, 72-80 (2011)), Sendai virus membrane fusion liposome (WO97/016171), modified liposome with a cell membrane permeable peptide, and the like. The antibody may be enclosed in the membrane fusion liposome for effective introduction into the cell. The enclosure of the peptide into the membrane fusion liposome is also encompassed in the “modification” of the present disclosure.

With respect to the cell membrane permeable peptide, various naturally occurring or artificially synthesized peptides have been reported so far (Joliot A. & Prochian. A., Nat Cell Biol. 2004; 6: 189-96). Non-limiting examples of cell membrane permeable peptides which may be suitable in the context of the present disclosure are set forth in table 1:

TABLE 1

| Name/ | | |
|---|---|---|
| protein source | Sequence | Reference |
| polyarginine | 5 to 20 arginine residues | Matsushita et al., (2003) |
| | | J. Neurosci.; 21, 6000-7 |
| Tat | RKKRRQRRR | Frankel et al., (1988) Cel. |
| | | 55, 1189-93., Green & |
| | | Loewenstein (1988) Cell 55, |
| | | 1179-88 |
| Penetratin | RQIKIWFQNRRMKWKK | Derossi et al., (1994) J. |
| | | Biol. Chem. 269, 10444-50 |
| Buforin II | TRSSRAGLQFPVCRVHRLLRK | Park et al., (2000) Proc. |
| | | Natl Acad. Sci. U.S.A. 97, |
| | | 8245-50 |
| Transportan | GWTLNSAGYLLGKINLKALAALAKKIL | Pooga et al., (1998) FASEB |
| | | J. 12, 67-77 |
| MAP (Model | KLALKLALKALKAALKLA | Oehlke et al., (1998) |
| Amphipathic | | Biochim. Biophys. Acta. |
| Peptide) | | 1414, 127-39 |
| K-FGF | AAVALLPAVLLALLAP | Lin et al., (1995) J. Biol. |
| | | Chem. 270, 14255-8 |
| Ku70 | VPMLK | Sawada et al., (2003) |
| | | Nature Cell Biol. 5, 352-7 |
| Ku70 | PMLKE | Sawada et al., (2003) |
| | | Nature Cell Biol. 5, 352-7 |
| Prion | MANLGYWLLALFVTMWTDVGLCKKRPKP | Lundberg et al., (2003) |
| | | Biochem. Biophys. Res. |
| | | Commun. 299, 85-90 |
| pVEC | LLIILARRIRKQAHAHSK | Elmquist et al., (2001) |
| | | Exp. Cell Res. 269, 237-44 |
| Pep-1 | KETWWETWWTEWSQPKKKRKV | Morris et al., (2001) |
| | | Nature Biotechnol. 19, |
| | | 1173-6 |
| SynB1 | RGGRLSYSRRRFSTSTGR | Rousselle et al., (200) |
| | | Mol. Pharmacol. 57, 679-86 |
| Pep-7 | SDLWEMMMVSLACQY | Gao et al., (2002) Bioorg. |
| | | Med. Chem. 10, 4057-65 |
| HN-1 | TSPLNIHNGQKL | Hong & Clayman (2000) |
| | | Cancer Res. 60, 6551-6 |

In another embodiment, the intracellular expression of the antibody may be obtained by using gene therapy. In other words, with administration of a gene delivery system to the subject, the gene delivery system including a nucleic acid molecule encoding the antibody or fragment thereof.

The term “gene therapy” typically refers delivery of nucleic acid molecules to cells in vivo using methods such as direct injection of DNA, receptor-mediated DNA uptake, viral-mediated transfection or non-viral transfection (for example, using a chitosan-based nanoparticle, e.g., as described in PCT/CA2016/050119) and lipid based transfection, all of which may involve the use of gene therapy vectors. Direct injection has been used to introduce naked DNA into cells in vivo (see e.g., Acsadi et al. (1991) Nature 332:815-818; Wolff et al. (1990) Science 247:1465-1468). A delivery apparatus (e.g., a “gene gun”) for injecting DNA into cells in vivo may be used. Such an apparatus may be commercially available (e.g., from BioRad). Naked DNA may also be introduced into cells by complexing the DNA to a cation, such as polylysine, which is coupled to a ligand for a cell-surface receptor (see for example Wu, G. and Wu, C. H. (1988) J. Biol. Chem. 263:14621; Wilson el al. (1992) J. Biol. Chem. 267:963-967; and U.S. Pat. No. 5,166,320). Binding of the DNA-ligand complex to the receptor may facilitate uptake of the DNA by receptor-mediated endocytosis. A DNA-ligand complex linked to adenovirus capsids which disrupt endosomes, thereby releasing material into the cytoplasm, may be used to avoid degradation of the complex by intracellular lysosomes (see for example Curiel el al. (1991) Proc. Natl. Acad. Sci. USA 88:8850; Cristiano et al. (1993) Proc. Natl. Acad. Sci. USA 90:2122-2126).

Defective retroviruses are well characterized for use as gene therapy vectors (for a review see Miller, A. D. (1990) Blood 76:271). Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines include psiCrip, psiCre, psi2 and psiAm. Retroviruses have been used to introduce a variety of genes into many different cell types, including epithelial cells, endothelial cells, lymphocytes, myoblasts, hepatocytes, bone marrow cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. Nos. 4,868,116; 4,980,286; PCT Application WO 89/07136; PCT Application WO 89/02468; PCT Application WO 89/05345; and PCT Application WO 92/07573).

Adeno-associated virus (AAV) may be used as a gene therapy vector for delivery of DNA for gene therapy purposes. AAV is a naturally occurring defective virus that requires another virus, such as an adenovirus or a herpes virus, as a helper virus for efficient replication and a productive life cycle (Muzyczka et al. Curr. Topics in Micro. and Immunol. (1992) 158:97-129). AAV may be used to integrate DNA into non-dividing cells (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). An AAV vector such as that described in Tratschin et al. (1985) Mol. Cell. Biol. 5:3251-3260 may be used to introduce DNA into cells (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790). Lentiviral gene therapy vectors may also be adapted for use in the invention.

General methods for gene therapy are known in the art. See for example, U.S. Pat. No. 5,399,346 by Anderson et al. (incorporated herein by reference). A biocompatible capsule for delivering genetic material is described in PCT Publication WO 95/05452 by Baetge et al. Methods of gene transfer into hematopoietic cells have also previously been reported (see Clapp, D. W., et al., Blood 78: 1132-1139 (1991); Anderson, Science 288:627-9 (2000); and, Cavazzana-Calvo et al., Science 288:669-72 (2000)).

The present disclosure also makes reference to fully human, humanized or chimeric immunoglobulin sequences. For example, the invention may include mouse immunoglobulin sequences or humanized mouse immunoglobulin sequences. The term “humanized” generally refers to a non-human polypeptide sequence that has been modified to minimize immunoreactivity in humans (e.g., framework and/or constant domain sequences), typically by altering the amino acid sequence to mimic existing human sequences, without substantially altering the function of the polypeptide sequence (see, e.g., Jones et al., Nature 321:522-525 (1986), and published UK patent application No. 8707252). Methods have been developed to replace light and heavy chain constant domains of the monoclonal antibody with analogous domains of human origin, leaving the variable regions of the foreign antibody intact. Alternatively, “fully human” monoclonal antibodies are produced in mice transgenic for human immunoglobulin genes. Methods have also been developed to convert variable domains of monoclonal antibodies to more human form by recombinantly constructing antibody variable domains having both rodent, for example, mouse, and human amino acid sequences. In “humanized” monoclonal antibodies, only the hypervariable CDR is derived from mouse monoclonal antibodies, and the framework and constant regions are derived from human amino acid sequences (see U.S. Pat. Nos. 5,091,513 and 6,881,557, each of which is incorporated herein by reference). It is thought that replacing amino acid sequences in the antibody that are characteristic of rodents with amino acid sequences found in the corresponding position of human antibodies will reduce the likelihood of adverse immune reaction during therapeutic use. A hybridoma or other cell producing an antibody may also be subject to genetic mutation or other changes, which may or may not alter the binding specificity of antibodies produced by the hybridoma.

Methods for producing polyclonal antibodies in various animal species, as well as for producing monoclonal antibodies of various types, including humanized, chimeric, and fully human, are well known in the art and highly predictable. For example, the following U.S. patents and patent applications provide enabling descriptions of such methods: U.S. Patent Application Nos. 2004/0126828 and 2002/0172677; and U.S. Pat. Nos. 3,817,837; 3,850,752; 3,939,350; 3,996,345; 4,196,265; 4,275,149; 4,277,437; 4,366,241; 4,469,797; 4,472,509; 4,606,855; 4,703,003; 4,742,159; 4,767,720; 4,816,567; 4,867,973; 4,938,948; 4,946,778; 5,021,236; 5,164,296; 5,196,066; 5,223,409; 5,403,484; 5,420,253; 5,565,332; 5,571,698; 5,627,052; 5,656,434; 5,770,376; 5,789,208; 5,821,337; 5,844,091; 5,858,657; 5,861,155; 5,871,907; 5,969,108; 6,054,297; 6,165,464; 6,365,157; 6,406,867; 6,709,659; 6,709,873; 6,753,407; 6,814,965; 6,849,259; 6,861,572; 6,875,434; 6,891,024; and 9,725,517, each of which are hereby incorporated by reference.

Moreover, the antibodies of the present disclosure may include fused immunoglobulin sequences, e.g. forming a multivalent and/or multispecific construct (for multivalent and multispecific polypeptides containing one or more V_{HH} domains and their preparation, reference is also made to Conrath et al., J. Biol. Chem., Vol. 276, 10. 7346-7350, 2001, as well as to for example WO 96/34103 and WO 99/23221), and immunoglobulin single variable domains comprising tags or other functional moieties, e.g. toxins, labels, radiochemicals, etc., which are derivable from the immunoglobulin single variable domains of the present disclosure.

Antibodies may be produced from any animal source, including birds and mammals. In addition, newer technology permits the development of and screening for human antibodies from human combinatorial antibody libraries. For example, bacteriophage antibody expression technology allows specific antibodies to be produced in the absence of animal immunization, as described in U.S. Pat. No. 6,946,546, which is incorporated herein by reference. Alternatively, antibody fragments and/or single chain antibodies may be synthetically produced in vitro. Alternatively, a nonrecombinant or recombinant antibody protein may be isolated from bacteria. An antibody or preferably an immunological portion of an antibody, can be chemically conjugated to, or expressed as, a fusion protein with other proteins.

“Treatment” and “treating” refer to administration or application of a therapeutic agent to a subject or performance of a procedure or modality on a subject for the purpose of obtaining a therapeutic benefit of a disease or health-related condition. For example, a treatment may include administration of a pharmaceutically effective amount of an antibody for prevention or treatment of a disease promoting apoptotic cell death in a subject.

As used herein, the terms “treatment”, “treating”, and the like, may include amelioration or elimination of a developed disease or condition once it has been established or alleviation of the characteristic symptoms of such disease or condition. As used herein, these terms may also encompass, depending on the condition of the subject, preventing the onset of a disease or condition or of symptoms associated with the disease or condition, including for example reducing the severity of the disease or condition or symptoms associated therewith prior to affliction with the disease or condition. Such prevention or reduction prior to affliction may refer, in the context of an immune disease or disorder, for example, a disease promoting apoptotic cell death, to administration of at least a pharmaceutically effective amount of an antibody to a subject that is not at the time of administration afflicted with the disease or condition. “Preventing” may also encompass preventing the recurrence or relapse of a previously existing disease or condition or of symptoms associated therewith, for instance after a period of improvement.

“Subject” and “patient” refer to either a human or non-human, such as primates, mammals, and vertebrates. In particular embodiments, the subject is a human.

The term “therapeutic benefit”, “therapeutically effective” or “pharmaceutically effective” as used throughout this application refers to anything that promotes or enhances the well-being of the subject with respect to the medical treatment of this condition. This includes, but is not limited to, a reduction in the frequency or severity of the signs or symptoms of a disease.

As used herein, the terms “pharmaceutically effective”, “therapeutically effective amount” and “effective amount” are used interchangeably to refer to an amount of a composition of the disclosure that is sufficient to result in the prevention of the development, recurrence, or onset of a disease or condition. For example, in certain embodiments these terms refer to an amount of a composition of the invention that is sufficient to result in the prevention of the development, recurrence, or onset of an immune disease or disorder, for example, a disease promoting apoptotic cell death, or one or more symptoms thereof, to enhance or improve the prophylactic effect(s) of another therapy, reduce the severity and duration of an immune disease or disorder, ameliorate one or more symptoms of an immune disease or disorder, prevent the advancement of an immune disease or disorder, cause regression of an immune disease or disorder, and/or enhance or improve the therapeutic effect(s) of additional an immune disease or disorder treatment(s).

A therapeutically effective amount can be administered to a patient in one or more doses sufficient to palliate, ameliorate, stabilize, reverse or slow the progression of the disease, or otherwise reduce the pathological consequences of the disease, or reduce the symptoms of the disease. The amelioration or reduction need not be permanent, but may be for a period of time ranging from at least one hour, at least one day, or at least one week or more. The effective amount is generally determined by the physician on a case-by-case basis and is within the skill of one in the art. Several factors are typically taken into account when determining an appropriate dosage to achieve an effective amount. These factors include age, sex and weight of the patient, the condition being treated, the severity of the condition, as well as the route of administration, dosage form and regimen and the desired result.

In one non-limiting embodiment, the present disclosure provides a kit which includes reagents that may be useful for implementing at least some of the herein described methods. The herein described kit may include at least one agent which is “packaged”. As used herein, the term “packaged” can refer to the use of a solid matrix or material such as glass, plastic, paper, fiber, foil and the like, capable of holding within fixed limits the at least one reagent. Thus, in one non-limiting embodiment, the kit may include the at least one agent “packaged” in a glass vial used to contain microgram or milligram quantities of the at least one agent. The kit can include optional components that aid in the administration of the therapeutic or pharmaceutical agents to patients, such as vials for reconstituting powder forms, syringes for injection, and customized delivery systems. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

As used herein and by way of example, and not by way of limitation, immune disease or disorder may refer to: rheumatoid arthritis, juvenile rheumatoid arthritis, osteoarthritis, psoriatic arthritis, diabetes mellitus, multiple sclerosis, encephalomyelitis, myasthenia gravis, systemic lupus erythematosus (SLE), autoimmune thyroiditis, atopic dermatitis, eczematous dermatitis, psoriasis, Sjogren's Syndrome, Crohn's disease, aphthous ulcer, iritis, conjunctivitis, keratoconjunctivitis, ulcerative colitis, inflammatory bowel disease (IBD), cutaneous lupus erythematosus, scleroderma, vaginitis, proctitis, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, interstitial lung fibrosis, Hashimoto's thyroiditis, autoimmune polyglandular syndrome, insulin-dependent diabetes mellitus, insulin-resistant diabetes mellitus, immune-mediated infertility, autoimmune Addison's disease, pemphigus vulgaris, pemphigus foliaceus, dermatitis herpetiformis, autoimmune alopecia, vitiligo, autoimmune hemolytic anemia, autoimmune thrombocytopenic purpura, pernicious anemia, Guillain-Barre syndrome, stiff-man syndrome, acute rheumatic fever, sympathetic ophthalmia, Goodpasture's syndrome, systemic necrotizing vasculitis, antiphospholipid syndrome or an allergy, Behcet's disease, severe combined immunodeficiency (SCID), recombinase activating gene (RAG 1/2) deficiency, adenosine deaminase (ADA) deficiency, interleukin receptor common g chain (c) deficiency, Janus-associated kinase 3 (JAK3) deficiency and reticular dysgenesis; primary T cell immunodeficiency such as DiGeorge syndrome, Nude syndrome, T cell receptor deficiency, MHC class II deficiency, TAP-2 deficiency (MHC class I deficiency), ZAP70 tyrosine kinase deficiency and purine nucleotide phosphorylase (PNP) deficiency, antibody deficiencies, X-linked agammaglobulinemia (Bruton's tyrosine kinase deficiency), autosomal recessive agammaglobulinemia, Mu heavy chain deficiency, surrogate light chain (g⁵/14.1) deficiency, Hyper-IgM syndrome: X-linked (CD40 ligand deficiency) or non-X-linked, Ig heavy chain gene deletion, IgA deficiency, deficiency of IgG subclasses (with or without IgA deficiency), common variable immunodeficiency (CVID), antibody deficiency with normal immunoglobulins; transient hypogammaglobulinemia of infancy, interferon g receptor (IFNGR1, IFNGR2) deficiency, interleukin 12 or interleukin 12 receptor deficiency, immunodeficiency with thymoma, Wiskott-Aldrich syndrome (WAS protein deficiency), ataxia telangiectasia (ATM deficiency), X-linked lymphoproliferative syndrome (SH2D1 A/SAP deficiency), diabetes or diabetic nephropathy, non-alcoholic fatty liver disease (NALFD) or non-alcoholic steatohepatitis (NASH), or hyper IgE syndrome.

The terms “determining,” “measuring,” “evaluating,” “assessing,” and “assaying,” as used herein, generally refer to any form of measurement, and include determining if an element is present or not in a biological sample. These terms include both quantitative and/or qualitative determinations, which both require sample processing and transformation steps of the biological sample. Assessing may be relative or absolute. The phrase “assessing the presence of” can include determining the amount of something present, as well as determining whether it is present or absent.

The expression “biological sample” includes in the present disclosure any biological sample that can be obtained from a subject as for example but without being limited thereto, blood and fractions thereof, urine, excreta, semen, tissue biopsies, tissue samples, seminal fluid, seminal plasma, prostatic fluid, pre-ejaculatory fluid (Cowper's fluid), pleural effusion, tears, saliva, sputum, sweat, biopsy, ascites, amniotic fluid, lymph, vaginal secretions, endometrial secretions, gastrointestinal secretions, bronchial secretions, breast secretions, and the like.

In one non-limiting embodiment, the herein described “biological sample” can be obtained by any known technique, for example by drawing, by non-invasive techniques, or from sample collections or banks, etc.

The term “contact” or “contacting” as used herein generally refers to placement in direct physical association, and includes both in solid and liquid form which can take place either in vivo or in vitro. Contacting generally includes contact between one molecule and another molecule, for example between a protein and an antibody. Contacting can also include contacting a cell or tissue, for example by placing a test agent in direct physical association with a cell or tissue (such as a biological sample) or by administration of an agent to a subject.

One of skill in the art will understand which standard controls or baseline levels are valuable in a given situation and be able to analyse data based on comparisons to standard control values. Standard controls and baseline levels are also valuable for determining the significance of data. For example, if values for a given parameter are widely variant in standard controls, variation in test samples will not be considered as significant.

Other examples of implementations will become apparent to the reader in view of the teachings of the present description and as such, will not be further described here.

Note that titles or subtitles may be used throughout the present disclosure for convenience of a reader, but in no way should these limit the scope of the invention. Moreover, certain theories may be proposed and disclosed herein; however, in no way they, whether they are right or wrong, should limit the scope of the invention so long as the invention is practiced according to the present disclosure without regard for any particular theory or scheme of action.

All references cited throughout the specification are hereby incorporated by reference in their entirety for all purposes.

It will be understood by those of skill in the art that throughout the present specification, the term “a” used before a term encompasses embodiments containing one or more to what the term refers. It will also be understood by those of skill in the art that throughout the present specification, the term “comprising”, which is synonymous with “including,” “containing,” or “characterized by,” is inclusive or open-ended and does not exclude additional, un-recited elements or method steps.

Unless otherwise defined, all technical and scientific terms used herein have the same meaning as commonly understood by one of ordinary skill in the art to which this invention pertains. In the case of conflict, the present document, including definitions will control.

As used in the present disclosure, the terms “around”, “about” or “approximately” shall generally mean within the error margin generally accepted in the art. Hence, numerical quantities given herein generally include such error margin such that the terms “around”, “about” or “approximately” can be inferred if not expressly stated.

Although various embodiments of the disclosure have been described and illustrated, it will be apparent to those skilled in the art in light of the present description that numerous modifications and variations can be made. The scope of the invention is defined more particularly in the appended claims.

### Methods

### Cell Culture and Plasmids

Primary and immortalized WT and PKM2-deficient MEFs (PKM2/A) were maintained in MEMa medium supplemented with 10% FBS, penicillin and streptomycin (Gibco-Invitrogen), and 0.1 mM of 2-mercaptoethanol (Lunt et al., 2015). HEK293T (293T) cells were maintained in DMEM containing 10% (vol/vol) FBS and antibiotics. pLHCX-Flag-mPKM2(K433E) (Plasmid #42514), pLHCX-Flag-mPKM2(C358S) (Plasmid #42513), pLHCX-Flag-mPKM2 (Plasmid #42512) were obtained from Addgene.

Lentiviral Infection with the scFv Library

Lentiviral particles were produced in 5×10⁷ 293T cells using pCMVD8.9 and pVSVg viral packaging vectors at a ratio of 1:1:1. For the first round of selection, culture supernatants containing lentiviral particles were collected, filtered, and used for infection of 1×10⁷ 293T cells per 10 mm plate. For the recloning step after rounds 2 and 3, 5×10⁶ cells were used. 48 h post-infection, the culture medium was replaced with fresh MEMa medium, supplemented with 10% FBS and penicillin/streptomycin (Gibco-Invitrogen).

### Intrabody Library Construction

The intrabody single-chain variable fragment (scFv) library was prepared using a naïve human combinatorial scFv phage library (Zhang et al., 2012). The scFv phagemid library was digested with SfiI, and the about 800-bp insert scFv coding sequence was ligated into the SfiI-digested lenti-viral vector driven by an EFla promoter (without a secretion leader sequence) followed by a FLAG tag.

### Selection of Intrabodies Conferring BimS Resistance in 293T Cells

Human BimS cDNA was subcloned into a pShooter™ mammalian expression vector (pCMV/myc/cyto; Invitrogen), to allow the expression of BimS driven by a CMV promoter. 5×10′ 293T cells were then infected with the intrabody library and then transfected with 4 μg/ml BimS plasmid, using 10 l of Lipofectamine® 2000 transfection reagent (Thermo Fisher). After 24 h post-infection, the culture medium was replaced with fresh MEMc medium supplemented with 10% FBS and penicillin/streptomycin (Gibco-Invitrogen).

Recovery of Selected scFv from the Genomic DNA by PCR and Construction of Intrabody Libraries for the Second and Third Rounds of Selection

The integrated intrabody coding sequences from the surviving cells were recovered after 48 h incubation and used to construct a secondary lentiviral library, as follows. Genomic DNA from the surviving 293T cells was recovered using a DNeasy™ Blood & Tissue kit (Qiagen). 100 ng of the genomic DNA was used as a PCR template. A pair of primers matching the regions flanking the scFv fragment was used to amplify the integrated antibody fragment from the genomic DNA. The PCR product was digested with SfiI and inserted back into the lentiviral vector for a subsequent round of BimS selection as described above. In total, over 300 clones with distinct DNA sequences were harvested and tested individually for the ability to confer BimS resistance. Sequences were analyzed with Vbase2.

Expression of scFv in E. coli

scFv coding sequences subcloned into pET28a plasmid were introduced into Rosetta™ (DE3)pLys cells (Novagen). Single colonies were picked and grown in 2 l of LB medium containing 50 μg/ml of kanamycin at 30° C. for 8 h, then incubated for 12 h at 4° C. with 0.2 mM IPTG under vigorous shaking. Cells were pelleted by centrifugation, frozen/thawed, resuspended in 50 ml of lysis buffer (Tris 25 mM pH 8.0, NaCl 300 mM), incubated 1 h on ice, and then lysed by sonication. The scFv was recovered from the soluble fraction by passage over a Ni⁺⁺-NTA affinity column (GE Healthcare).

### Target Protein Immunoprecipitation

Flag-tagged intrabody was introduced along with a tandem Strep-tag by PCR into the same lentiviral vector used for selection. 293T cells infected with the intrabody lentivirus were incubated at 30° C. for 72 h as described above. After washing with cold PBS, 5×10⁸ cells were lysed for 15 minutes on ice in lysis buffer (50 mM Tris HCl, pH 7.4, 150 mM NaCl, 1 mM EDTA, 1% Triton™ X-100). Cell lysates were clarified by centrifugation for 15 minutes at 4° C. at 16,000×g. The total protein content of the soluble fraction was quantified using the BCA assay. For pull-down experiments, 10 mg of protein lysate was incubated with 200 al of EZview™ Red anti-FLAG® M2 Affini-ty Gel (Sigma-Aldrich) for 2 h at 4° C. Beads were washed 3 times in wash buffer (50 mM Tris HCl, pH 7.4, 150 mM NaCl, 1 mM EDTA, 0.1% Triton X-100). Elution was performed under native conditions by competition with 3×FLAG peptide following the manufacturer's protocol. Eluates were used for the second step purification using Strep-Tactin Superflow Plus™ [Qiagen], following the manufacturer's instructions. The final two-step purified protein was used for SDS-PAGE analysis. Bands of interest were cut out from the gel and subjected to in-gel digestion with trypsin (Promega, Fitchburg, Wis., USA) followed by MALDI TOF/TOF mass spectrometry analysis (Biomolecular and Proteomics Mass Spectrometry Facility, University of California at San Diego).

### Cell Viability and Clonogenic Survival Assays

Cell viability was measured with a Countess™ Automated Cell Counter (Invitrogen) using trypan blue. For the clonogenic survival assay, 293T or MEFs cells were seeded in 6-well plates at 1×10⁵ cells/well in a 2-ml volume, transfected with BimS cDNA and incubated for 24 h. Afterwards, the medium was replaced and the cells cultured for 3-4 days. Thereafter, the plates were rinsed with PBS and fixed and stained with a solution containing crystal violet (0.5% w/v) and glutaraldehyde (6% v/v) as described (Franken et al., 2006). The results were quantified using ImageJ software (either total area or number of colonies, as indicated in the figure legends).

### PIKM2 Protein Expression and Purification

pET28a-His-hPKM2 plasmid was obtained from Addgene. The PIKM2 mutants, pET28aHis-hPKM2(C358S), pET28a-His-hPKM2(K270M) were generated by Quick-Change mutagenesis (Stratagene). All plasmids were verified by DNA sequencing and transformed into Escherichia coli strain BL21(DE3). WT and mutant PKM2 proteins were overexpressed in LB medium at 30° C. with 200 mM IPTG for 3 h. Cells were harvested and lysed in buffer containing 25 mM Tris (pH 8.0), 300 mM NaCl. The supernatants were loaded on a Ni⁺⁺-NTA affinity column (GE Healthcare) for protein purification.

### Pyruvate Kinase Assay

Pyruvate kinase activity was measured by using Kinase-Glo® Plus Luminescent Kinase Assay kit (Promega). 50-100 nM of purified WT or mutant PKM2 was added in 100 μl assay buffer containing 50 mM Tris pH7.5, 100 mM KCl, 10 mM MgCl₂, 200 μM PEP, 200 μM ADP, and 3% DMSO. After a 15-min incubation, Kinase-Glo Plus™ reagent (Promega Corporation, Madison, Wis., USA) was added, according to the manufacturer's instructions. In some cases, 0-40 μM fructose 1,6-bisphosphate (FBP) and 0-150 nM scFv 5 were added.

### Mitochondria Isolation and Cytochrome c Release Assay

Mitochondria were isolated from 5×10⁸ cells as described (Waterhouse et al., 2001). The freshly isolated mitochondria (100 mg protein/ml) were then incubated with recombinant cleaved Bid protein (Kuwana et al., 2002) at the indicated concentrations in the presence or absence of purified scFv #5. After incubation for 30 min at 37° C., mitochondria were collected by centrifugation at 10 000×g and analyzed by immunoblotting as described (Waterhouse et al., 2001).

### Immunoblots of Bcl-2 Family Proteins

293T cells were washed with PBS and lysed with PBS containing 1% NP-40. Protein concentration was determined using Pierce™ BCA reagent (ThermoFisher; 23221, 23224). 35 μg protein was loaded in each lane of 12% SDS-PAGE gels. Proteins were transferred to nitrocellulose membrane and immunoblotted with the following primary antibodies: anti-Bax antibody (Santa Cruz N20), anti-Bak antibody (Cell Signaling 3814), anti-Bid antibody (R&D Systems AF860), anti-Bim antibody (Sigma B7929), anti-Puma antibody (Cell Signaling 4976), anti-Bcl-2 antibody (Abcam 32124), anti-Bcl-xL antibody (Cell Signaling 2764) and anti-Mcl-1 (Santa Cruz S19) at 1:1000 dilution. The secondary anti-rabbit and mouse antibodies, conjugated with HRP, were obtained from Santa Cruz and were used at 1:2000 dilution. The luminescence signal was detected using ECL reagent (ThermoFisher 32106).

siRNA Silencing of PKIM2

Untreated or IB5-infected 293T cells (5×10⁵ per well) were seeded into 6-well plates (Falcon) and transfected with 30 nM siRNA. Lipofectamine™ 2000 (Invitrogen, Carlsbad, Calif., USA) was used for transient transfection according to the manufacturer's protocol. After a 30-h incubation, fresh medium containing 30 nM siRNA and 4 μg/ml BimS expression plasmid was added. Cell viability assay was assayed after a further 36-h incubation. The PKM2-siRNA and control siRNA were purchased from Dharmacon (SiGENOME SMART™ pool hPKM2, Si156, and ONTARGETplus™ non-targeting siRNA #2) (Goldberg and Sharp, 2012).

### Results

Molecules regulating the core apoptotic machinery could be important in a variety of physiological situations. To discover such proteins, the present inventors adapted a functional selection approach that had been used to identify proteins that participate in various intracellular functions (Xie et al., 2014; Zhang et al., 2011; Zhang et al., 2012). First, the present inventors infected HEK293T (293T) cells with a lentiviral library of genes encoding “intrabodies” (Zhang et al., 2012): intracellularly expressed single chain antibodies (scFv). These scFv included the variable regions from immunoglobulin heavy (V_{H}) and light (V_{L}) chains, connected by a flexible peptide linker, which formed a naïve human combinatorial scFv lentiviral library (diversity 4.5×10⁹). The present inventors induced apoptosis in the cells by transiently transfecting them with a cDNA encoding BimS, the most potent pro-apoptotic isoform of Bim. Bim is one of the most important Bcl-2-family proteins in the BH3-only category and is required for cell homeostasis in numerous physiological settings. Importantly, BimS promotes cell death by acting in the central apoptotic death mechanism, both by activating Bax/Bak and by sequestering anti-apoptotic Bcl-2 family members (Chen et al., 2005; Kuwana et al., 2005b). Bax/Bak activation then directly produces MOMP, crista junction remodeling, and apoptosis (Kuwana et al., 2002; Yamaguchi et al., 2008).

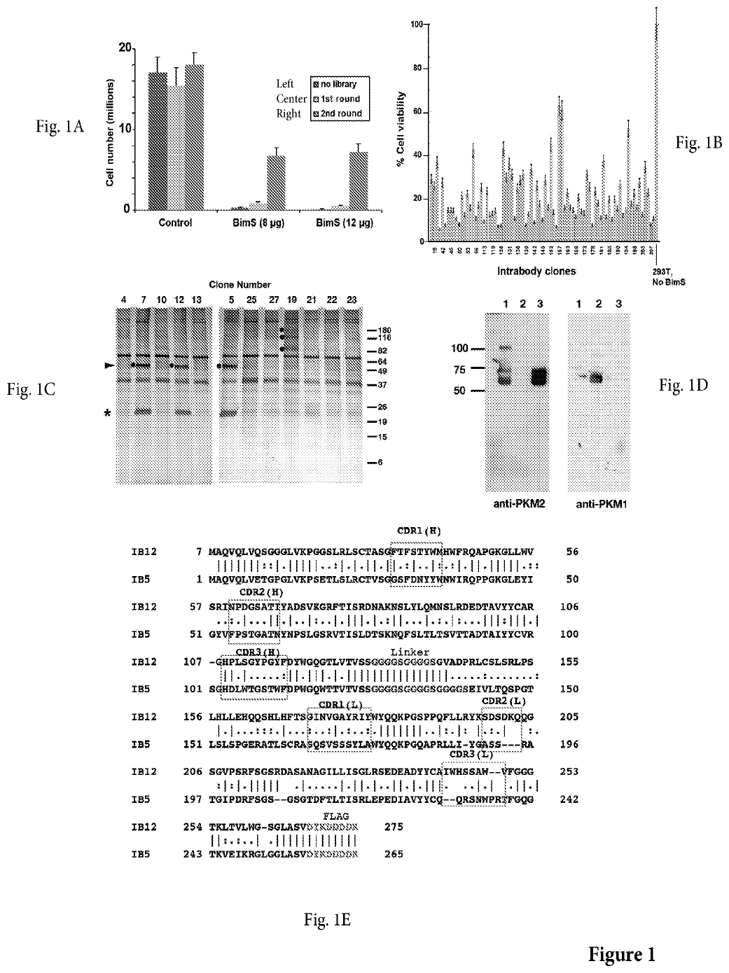

The present inventors then selected for intrabodies that rescued cells from BimS-induced death, by recovering scFv-encoding DNA from the surviving cells. This selection process was efficient, as BimS transfection killed about 99% of the control cells, whereas expression of the lentiviral scFv library rescued a small percentage of the cells (FIG. 1A). The present inventors then recovered the scFv-encoding DNA from surviving cells with which the present inventors created a new lentiviral library for a second round of selection. In this round, intrabodies rescued about 40% of the cells from BimS-induced death, implying a substantial enrichment of intrabodies with pro-survival activity (FIG. 1A). A third round of selection did not increase the percentage of cell survival.

To isolate single intrabody-encoding genes, the present inventors subcloned the enriched DNA from the second round into bacteria. Next, the present inventors introduced about 300 of these individual scFv genes separately into 293T cells. Many of the intrabodies rescued 293T cells from apoptosis induced by BimS expression, to varying extents (FIG. 1B). To identify protein targets, the present inventors performed FLAG-pull-downs of some of the intrabody-target protein complexes, which the inventors resolved on silver-stained SDS-polyacrylamide gels. The present inventors found that some intrabodies precipitated specific cellular proteins (FIG. 1C). By MALDI-TOF mass spectrometry and immunoblot analysis (FIG. 1D), the present inventors identified one protein target of three different scFv-encoding DNA clones (5, 7 and 12) as the M2 isoform of pyruvate kinase (PKM2). scFvs 5 and 7 had an identical DNA sequence (not shown), whereas scFv 12 was different (the light chain CDRs of scFv 12 were essentially distinct from those of scFv 5, and heavy chain CDRs were only about 30% identical; FIG. 1E). This apparent convergent selection underscores the potential importance of PKM2 as an apoptosis-regulating target protein.

In the following sections, intrabody 5 (hereafter referred to as IB5) is used as an illustrative example of the principle of the invention, but it is not intended to limit the present invention.

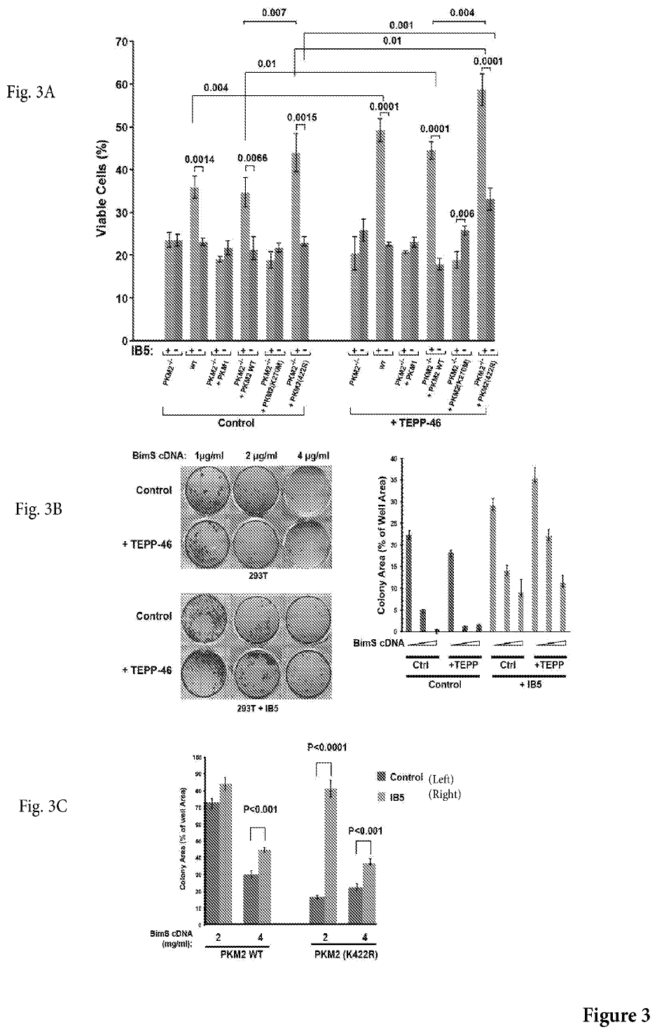

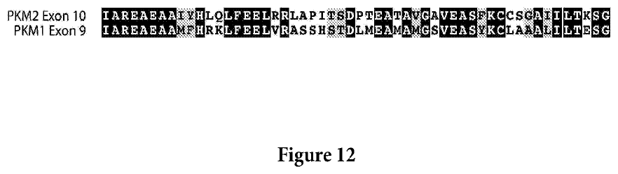

In vitro experiments (FIG. 3A, 3B and not shown) revealed that IB5 bound directly to PKM2 but not PKM1, suggesting that intrabody binding to PKM2 involves an epitope that includes residues at least encoded by the nucleic acid sequence contained in exon 10 (SEQ ID NO: 15), which is unique to PKM2 (FIG. 12). For example, the epitope may include at least a portion of the amino acid sequence which is encoded by the nucleic acid sequence contained in exon 10. Alternatively, the epitope may be formed by at least amino acids that are specifically encoded by the nucleic acid sequence contained in exon 10, where the specific characteristic is determined relative to the amino acid sequence which is encoded by the nucleic acid sequence contained in exon 9 (SEQ ID NO: 16), namely the 22 amino acid residues of exon 10 (FIG. 12 Alternatively, the epitope may include a mixture of residues that are specifically encoded by the nucleic acid sequence contained in exon 10 and residues that are common to exon 9 and exon 10. Alternatively, the epitope may include a conformational epitope which includes amino acid residues encoded by the nucleic acid sequence contained in exon 10. Alternatively, the epitope may exclude at least a portion of the amino acid sequence which is encoded by the nucleic acid sequence contained in exon 9.

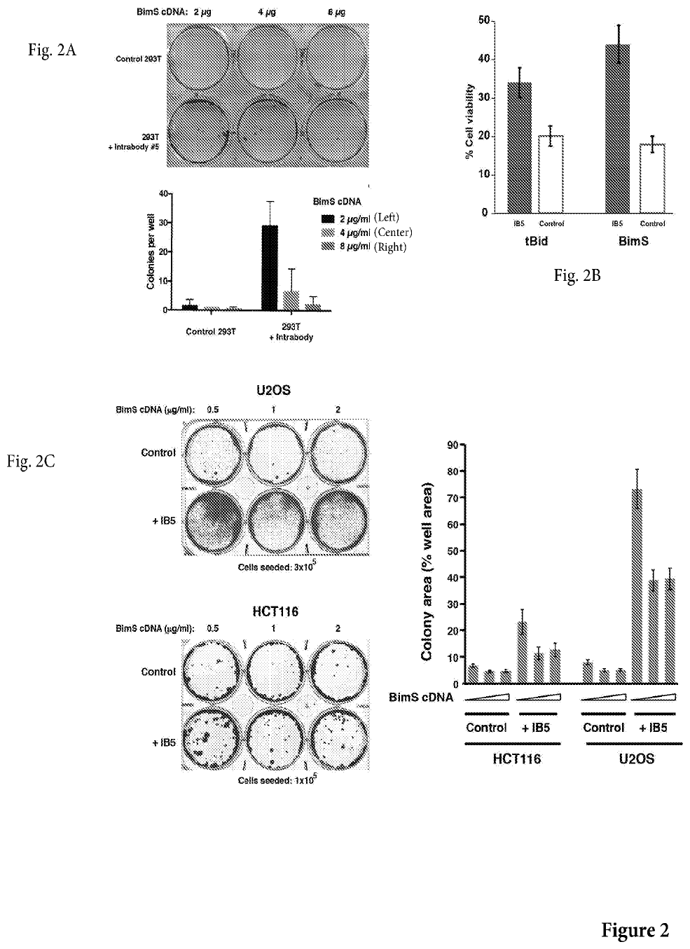

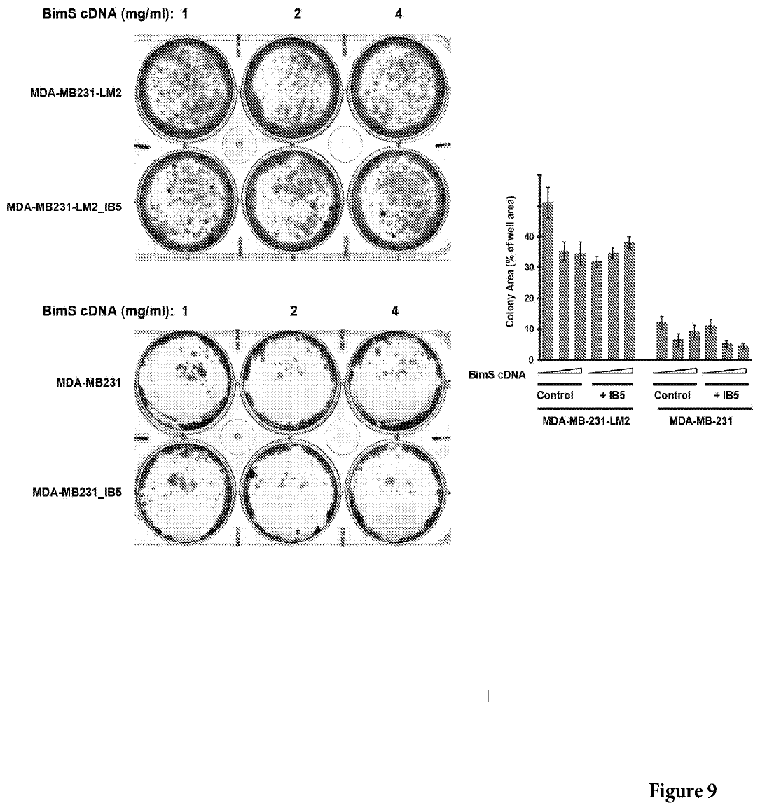

IB5, like a number of the present inventors intrabody hits, rescued a moderate percentage (about 15-20%) of the 293T cells from BimS-induced death. The present inventors suspect that it is a tall order for intrabodies to be very potent. These molecules are monovalent and thus do not have the enhanced avidity of IgG or IgM. Also, to have an effect revealed by selection, they would likely need to be expressed as abundantly as their target proteins, and therefore might be present in limiting amounts. Consistent with this possibility, the present inventors expressed IB5 with a weaker promoter and found that its survival effect was reduced (not shown). In any case, this cell survival is potentially significant, given how potently and directly BimS activates the central mitochondrial pathway. Importantly, the present inventors found that the PKM2-specific intrabody produced clonogenic survival, meaning that the surviving cells were able to proliferate (FIGS. 2A, 2C and 3B). Thus, this latent anti-apoptotic activity of PKM2 could be consequential for physiological situations, e.g. for the progression of pre-neoplastic cells. If even a fraction of these survive, they could ultimately undergo further adaptations, leading to oncogenesis. In further clonogenicity experiments, the present inventors found that IB5 substantially protected other tumor cell lines, U2OS and HCT116, from BimS-induced apoptosis (FIG. 2C). However, anti-apoptotic function was cell typespecific, as IB5 failed to rescue two breast cancer-derived cell lines (parental MDA-MB231 and a lung metastatic derivative, MDA-MB231-LM2), from BimS-induced cell death (FIG. 9).

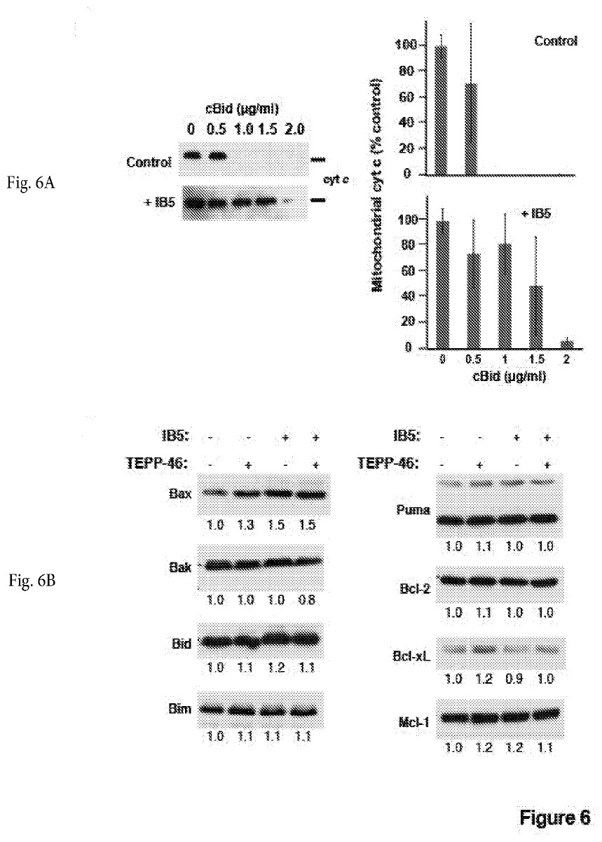

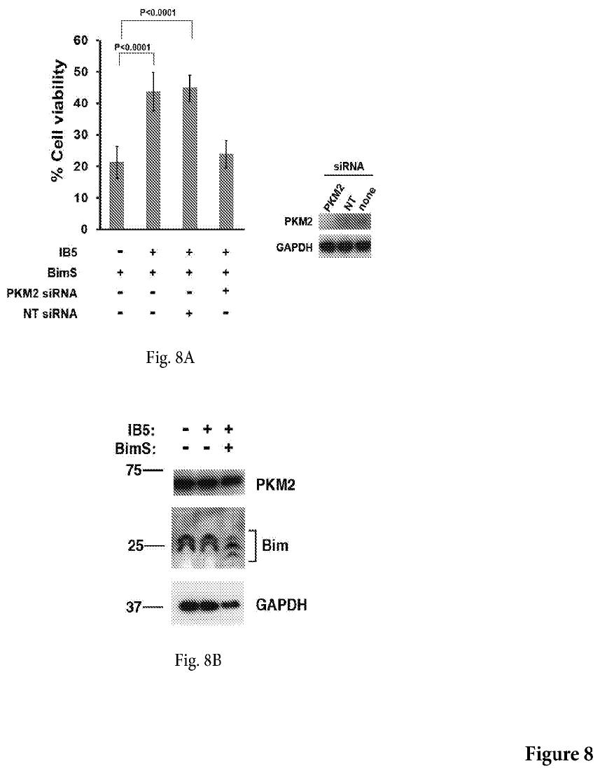

To verify that PKM2 is indeed the functional target of IB5, the present inventors used siRNA to silence PKM2 in 293T cells and saw a substantial reduction in the ability of IB5 to rescue cells from BimS killing (FIG. 8A). The present inventors confirmed this result using MEFs genetically deficient for PKM2 (Lunt et al., 2015). Human and mouse PKM2 display about 98% sequence identity, and thus IB5 was expected to cross-react with the mouse protein. Indeed, IB5 rescued a percentage of the WT cells from BimS-induced killing, but this rescue was entirely abrogated in PKM2-deficient MEFs (FIGS. 3A and 6A). Moreover, reconstituting the PKM2-null MEFs with cDNA encoding WT PKM2, but not PKM1, restored the cytoprotective ability of IB5 (FIG. 3A). PKM2-null MEFs are known to upregulate expression of PKM1 (Lunt et al.), further confirming the specificity of the effect for PKM2. The present inventors conclude that IB5's anti-apoptotic function is not recapitulated by the ablation of PKIM2, but rather requires the presence of PKM2. Therefore, IB5 acts positively on PKM2 to activate a cytoprotective function. The intrabody IB5 likely mimics an unidentified physiological interaction partner of PIKM2 that activates this anti-apoptotic function.

The present inventors ruled out the explanation that IB5 expression could alter the intracellular levels of PIKM2 (FIG. 8B). The present inventors could not determine whether IB5 expression altered the expression level of the exogenous BimS cDNA, because the control condition, in which IB5 was absent, produced cell death in almost all of the BimS-expressing cells. However, this is unlikely, as IB5 expression did not affect levels of endogenous Bim EL and L isoforms in normal 293T cells (FIG. 8B). Moreover, Bim EL, L and S isoforms were detectable in the BimS-resistant cell population rescued by IB5.

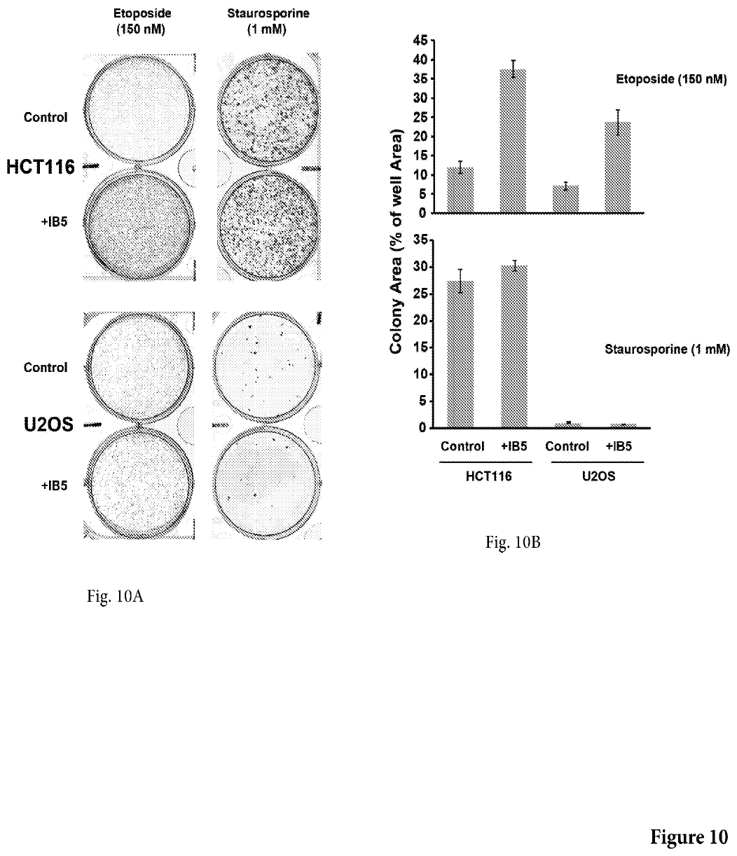

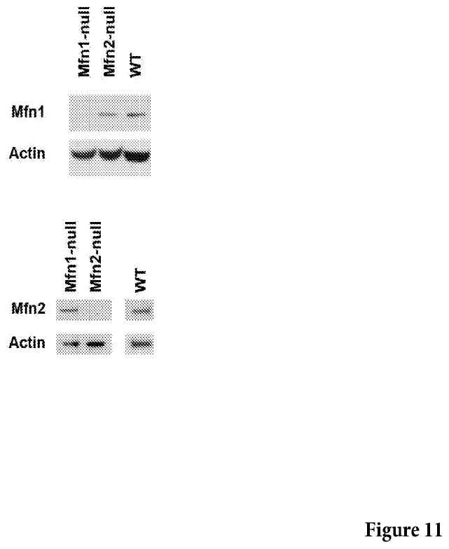

The present inventors also found that the PKM2-specific intrabody protected cells from death induced by another potent BH3-only protein, tBid (FIG. 2B). As Bim and Bid are two major activators of the core apoptotic pathway, directly upstream of Bax/Bak activation and mitochondrial permeabilization, this suggests that PIKM2 can inhibit the common apoptotic pathway at the level of Bax/Bak activation. If so, PKIIM2 could oppose physiological cell death triggered by some proapoptotic pathways. Indeed, the inventors found that IB5 promoted clonogenic survival in cells treated with the DNA-damaging drug, etoposide, although not with another cytotoxic agent, staurosporine (FIG. 10).

Based on the foregoing results, the reader will understand that similar cytoprotective function(s) may be reasonably expected with at least some of the other clones isolated with the herein described method, including at least IB12.

### Enhanced PIKM2 Glycolytic Activity is not the Sole Explanation for the Anti-Apoptotic Effect of IB5

Because glucose metabolism can influence cell survival (Fulda and Debatin, 2007; Moley and Mueckler, 2000; Munoz-Pinedo et al., 2012), The present inventors asked whether IB5 could rescue cells simply through stimulating PKM2's glycolytic activity. First, to analyze the antibody's interaction with PKM2, the present inventors produced monovalent scFv 5 and PIKM2 as recombinant proteins in E. coli and analyzed mixtures of these proteins, by blue native gel electrophoresis (FIG. 3A). The present inventors found that monovalent scFv 5 strongly increased the tetrameric PIKMV2 species and shifted up the tetramer band to a degree that was dependent on the molar input ratio of scFv. Thus, the intrabody bound directly to PKM2, promoting its stable tetramerization, and moreover, higher input ratios of scFv:PKIM2 produced increased binding stoichiometry. In contrast, scFv 5 failed to shift the electrophoretic mobility of recombinant PKM1 (which is constitutively tetrameric), confirming the antibody's specificity for PIKM2.

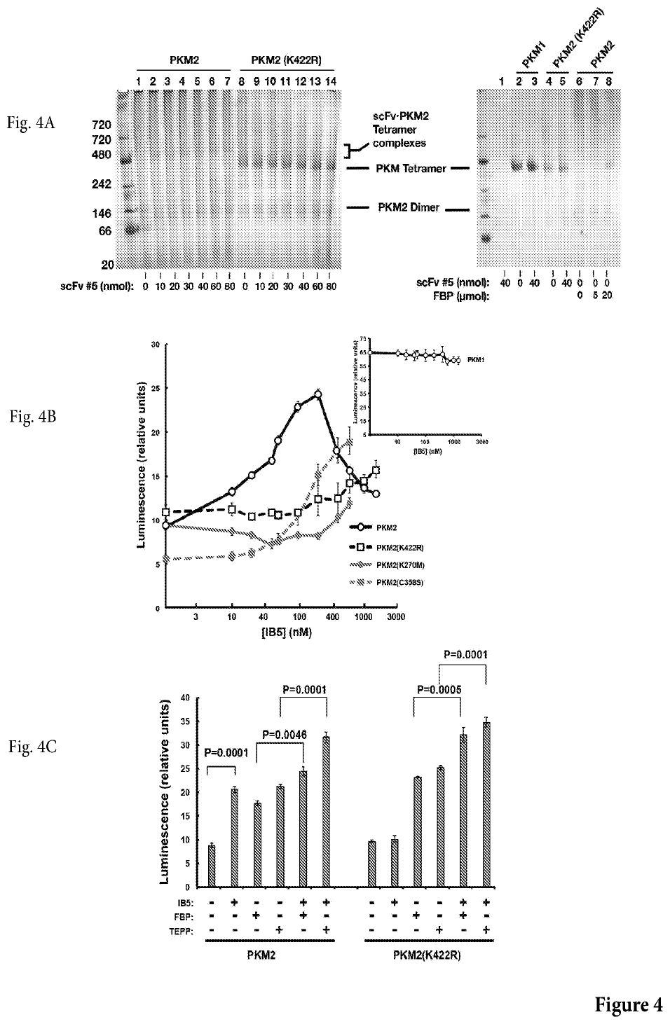

The present inventors then found that purified scFv 5 stimulated PKM2's glycolytic activity in a concentration-dependent manner (FIG. 4B), confirming indirectly that the scFv interacts with PKM2. (For reasons still unclear, the curve was biphasic: pyruvate kinase activity reached a maximum and declined at higher concentrations of scFv 5.) This result suggests that scFv 5 activates PKM2 allosterically. Considering this, the present inventors initially hypothesized that the anti-apoptotic effect of IB5 purely reflected an increased pyruvate kinase activity. However, further experiments challenged this idea. The present inventors found that treating cells with the PKIM2-activating compounds TEPP-46 (FIG. 3C) or DASA-58 (not shown; Anastasiou et al., 2012; Boxer et al., 2010; Jiang et al., 2010) alone did not protect cells from BimS-induced death. Similarly, reconstituting PKM2-null MEFs with the constitutively active M1 isoform of PKIM did not rescue cells (FIG. 3A). The present inventors conclude that high pyruvate kinase activity alone is insufficient to rescue cells from BimS-induced apoptosis.

However, culturing 293T cells in the presence of TEPP-46 (FIG. 2C) or DASA-58 (not shown) enhanced the pro-survival effect of IB5 expression to a modest but statistically significant extent. Thus, whereas high PK activity by itself was insufficient to produce an anti-apoptotic effect, chemical activation of PKM2 did slightly enhance IB5's effect. This could mean that increased glycolytic activity does contribute to PKM2's cell survival function. Alternatively, the effect of IB5, alone or in combination with TEPP-46, might result from the stabilization of a tetrameric conformation of PKM2, independent of PKM2's glycolytic activity.

Studies with PKM2 Mutants

To begin to define the aspects of PKM2 function required for the IB5-induced anti-apoptotic effect, the present inventors reconstituted PKM2-null MEFs with WT or mutant forms of PKM2. The present inventors first analyzed the K270M mutation, reported to be catalytically dead (Bollenbach et al., 1999; Dombrauckas et al., 2005; Luo et al., 2011). While this mutant by itself indeed lacked basal glycolytic activity in vitro, the addition of high concentrations of scFv 5 increased the catalytic activity of this mutant (FIG. 4B). Whereas WT PKM2 restored the cytoprotective effect of IB5 in reconstituted MEFs, PKM2 (K270M) did not. If the K270M mutation merely inactivated the catalytic site, this would suggest that PKM2's glycolytic activity is required for the cell survival effect. However, using blue native gel electrophoresis, the present inventors found that the K270M mutation also prevented the protein from forming stable tetramers in vitro, when incubated either with scFv 5 or with FBP (not shown). These results have at least three possible explanations (which are not mutually exclusive): 1) PKM2's glycolytic activity is required for the anti-apoptotic function; 2) a specific tetrameric conformation of PKM2 that produces a nonglycolytic activity is required; or 3) IB5 binding is reduced by the K270M mutation. The present inventors note that the stimulation of this mutant's glycolytic activity seen at high concentrations of scFv 5 (FIG. 4B) suggests that IB5 has some affinity for PKM2 (K270M).

To test the possibility that PKM2 tetramerization is important for the cell survival activity, the present inventors reconstituted PKM2-deficient MEFs with the stably tetrameric PKIM2 (K422R) mutant. This mutant is glycolytically inactive, unless an allosteric activator such as FBP is added, causing a quaternary conformational change from T-state to R-state (Wang et al., 2015a). Blue native gel electrophoresis confirmed the spontaneous formation of K422R tetramers, in the absence of FBP (FIG. 4A). The addition of scFv 5 shifted the tetramer band up only in the presence of allosteric activator FBP (FIG. 4A, right panel), suggesting that scFv 5 binds with higher affinity to the active R-state tetramer. Also, recombinant scFv 5 stimulated the glycolytic activity of PKM2 (K422R) in a concentration-dependent manner (FIG. 4B). At high concentrations of scFv 5, the mutant's activity was at least equal to that of WT PKM2. The present inventors conclude that IB5 can bind to PKIM2 (K422R), at least to the active R-state form. Interaction of IB5 may further stabilize the active R-state conformation of PKM2, especially in the presence of the allosteric activators FBP or TEPP-46.

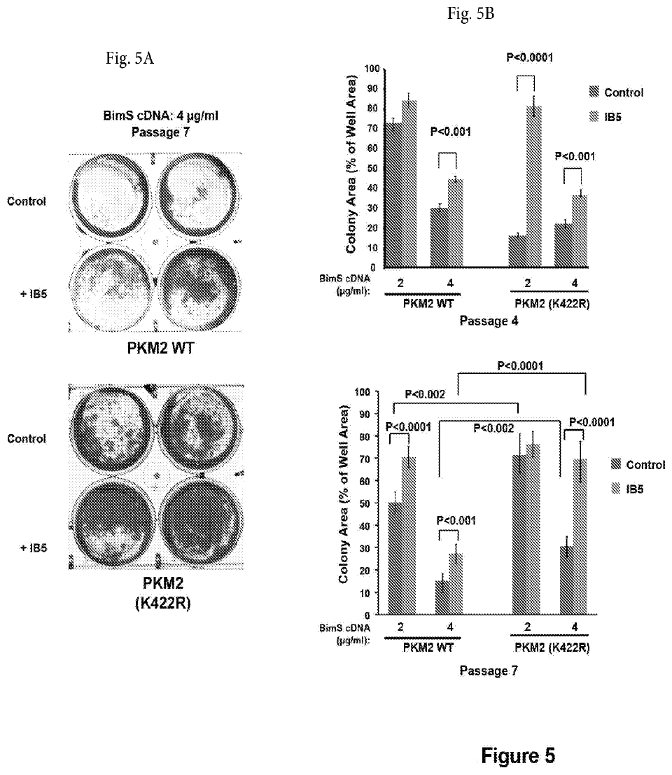

In reconstituted early-passage MEFs, PKM2 (K422R) slightly increased the numbers of viable cells compared with WT PKM2, and IB5 expression further increased viability (FIG. 3A). At this early passage, IB5 expression enhanced clonogenic survival with PKM2 (K422R) in a manner similar to WT PKM2 (FIG. 5B). (For unknown reasons, survival in the absence of IB5 was somewhat reduced with this mutant, compared with WT PKM2). However, in later-passage MEFs, the K422R mutant promoted clonogenic survival even in the absence of IB5, and survival was further enhanced by IB5 expression (FIG. 5A, B). This suggests that tetrameric PKM2 promotes a cell survival function that develops over time (see below).

The nuclear form of PIKM2 is thought to be dimeric, whereas tetramers are restricted to the cytoplasm (Gao et al., 2012). If so, the present inventors' data imply that the anti-apoptotic effect of IB5 involves cytoplasmic PIKM2 molecules and does not require the transcriptional activities ascribed to dimeric PIKM2 in the nucleus. Consistent with this, the present inventors found that the K270M mutant, which does not form stable tetramers visible in blue-native gels even in the presence of FBP (not shown) but is reportedly competent in nuclear transactivational activity (Luo, 2011 #352), failed to support IB5-induced cell rescue (FIG. 3A).

### A Mitochondrial Role in IB5-Induced Apoptosis Resistance

Because no cytoprotective effect was seen when PKM2 was stimulated with allosteric activator TEPP-46 alone, or when PKM2 was replaced with the constitutively active PKIM1 (FIG. 3), the present inventors conclude that increased pyruvate kinase activity per se was insufficient for the cell survival effect induced by IB5. To test whether IB5:PIKM2 complexes could affect pro-apoptotic Bcl-2 family proteins directly, the present inventors used their own previously well-validated in vitro systems that recapitulate Bcl-2 family protein function in membranes. Here, recombinant Bax and cleaved Bid (cBid) proteins are incubated with protein-free liposomes or with isolated Xenopus egg mitochondrial outer membrane vesicles. Bax then becomes inserted into the membranes and forms large pores that recapitulate MOMP as it occurs within cells (Gillies et al., 2015; Kuwana et al., 2002; Kuwana et al., 2016; Schafer et al., 2009). The present inventors observed no effect of adding recombinant scFv 5 and PKIVI2 to these systems (not shown). Thus, the inventors saw no evidence that PKM2 acts directly on the process of Bax/Bak-mediated MOMP.

On the other hand, mitochondria isolated from 293T cells expressing IB5 were reproducibly more resistant than control mitochondria to MOMP induced by treatment with cleaved Bid protein (FIG. 6A). This suggests that mitochondrial changes could explain the cellular resistance to apoptosis induced by IB5 (FIG. 2). As Bcl-2 family proteins are the most prominent regulators of apoptotic death at mitochondria, the present inventors first considered whether altered levels of these proteins could be responsible for MOMP resistance. However, the present inventors found that IB5 expression and TEPP-46 treatment (alone or in combination) failed to change the mitochondrial levels of the major family members Bax, Bak, Bid, Bim, Puma, Bcl-2, Bcl-xL and Mcl-1 (FIG. 6B).

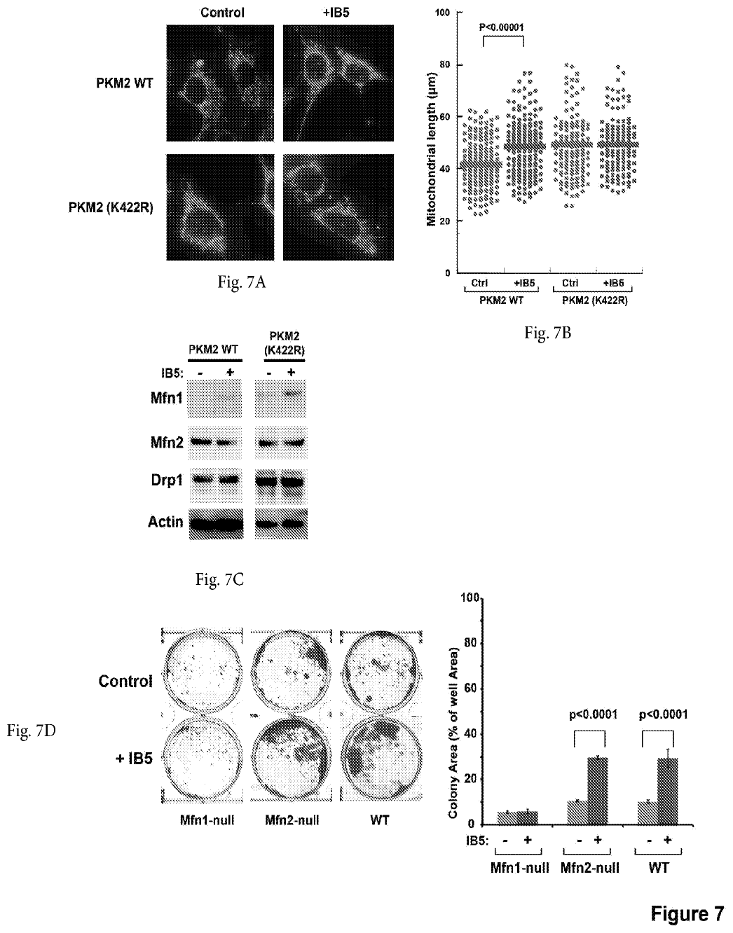

The present inventors next used microscopy to analyze the effect of IB5 and PIKM2 on mitochondrial morphology. The present inventors found that, in PKM2-null MEFs reconstituted with WT PIKM2, IB5 expression increased the average mitochondrial length (FIG. 7A,B). Furthermore, reconstitution of MEFs with PKM2 (K422R) by itself produced a similar mitochondrial lengthening, even without IB5. These results raised the possibility that PKM2-dependent mitochondrial lengthening and cell rescue from BimS expression could involve alterations of proteins that regulate mitochondrial dynamics. In this regard, a recent study reported that PIKM2 overexpression promoted mitochondrial fusion by binding to p53 and MDM2, promoting p53 ubiquitination and degradation, and thereby inhibiting expression of Drp1, a protein required for mitochondrial fission (Wu et al., 2016). However, the PKM2-dependent cytoprotective effect described here was not accompanied by changes in the levels of p53 (not shown) or Drp1 (FIG. 6C), in MEFs reconstituted with PKM2 WT or PKM2 (K422R).

The present inventors did find that IB5 expression substantially increased the levels of Mfn1, a protein involved in mitochondrial fusion (FIG. 7C), but left Mfn2 levels unchanged. Importantly, reconstituting MEFs with PKM2 (K422R) alone, in the absence of IB5, increased Mfn1 levels. Furthermore, IB5 expression in cells expressing the K422R mutant upregulated Mfn1 even further (FIG. 7C). To determine whether Mfn1 is required for the cytoprotective effect of IB5 and PIKM2, the present inventors measured BimS-resistant clonogenic survival in WT, Mfn1-deficient and Mfn2-deficient MEFs. Western blots confirmed the deletions of Mfn1 or Mfn2 (Supplementary FIG. 4). As FIG. 7D shows, IB5 failed to rescue Mfn1-deficient MEFs from BimS-induced clonogenic death. Even when IB5 was not expressed, Mfn1-null MEFs showed greater sensitivity to BimS-induced apoptosis than WT MEFs. In contrast, Mfn2-null MEFs responded similarly to WT. A previous study reported that Mfn1 directly inhibits mitochondria-mediated apoptosis at the step of Bax activation (Ryu et al., 2012), downstream of Bax mitochondrial translocation. However, the present inventors' results appear inconsistent with this mechanism, as PIKM2 (K422R) by itself upregulated Mfn1 and produced an increase in mitochondrial length, but did not alter the cells' sensitivity to BimS-induced death in early passage MEFs.

The present inventors did find that MEFs expressing PKM2 (K422R), upon extended passaging, developed a significant resistance to BimS-induced death, even in the absence of IB5 (FIG. 5). The present inventors hypothesize that over time Mfn1 upregulation opposes apoptosis to some extent by enhancing mitochondrial fusion. This could be expected to gradually improve the overall health of the mitochondrial network and may, for example, curtail the production of reactive oxygen species. The present inventors previously observed a similarly delayed, but detrimental, effect on mitochondrial function in MEFs haploin sufficient for the Opal protein. In that case, the late-passage cells displayed a decrease in respiratory function that likely resulted from inefficient mitochondrial fusion. Nevertheless, despite the upregulation of Mfn1 seen in both early- and late-passage cells expressing PKM2 (K422R), IB5 expression enhanced cell survival. This implies that Mfn1 upregulation only partly explains the cytoprotective function of PKM2 activated by IB5. In conclusion, the IB5*PKM2 interaction opposes apoptosis both by upregulating Mfn1 and by promoting another unidentified PKM2-dependent mechanism.

How IB5 cooperates with PKM2 to upregulate Mfn1 is unknown. One possibility is that IB5, by driving PKM2 molecules into the tetramer form, could reduce the amount of dimeric nuclear PKM2 and thereby abrogate transcriptional functions of PKM2 that could downregulate Mfn1. Alternatively, PKM2 tetramers could act in the cytoplasm to regulate the postsynthetic modification or degradation of Mfn1. One study reported that after phosphorylation by ERK, Mfn1 produces an effect opposite to that seen for Mfn1 in the present inventors experiments; that is, phosphorylated Mfn1 binds more tightly to Bak, producing cell death (Pyakurel et al., 2015). Another group reported that Mfn1 is subject to proteasomal degradation involving the E3 ligase MARCH5, leading to increased apoptosis (Choudhary et al., 2014). Tetrameric PKM2 might inhibit either of these processes.

In summary, the present inventors' data show that high pyruvate kinase activity by itself was insufficient to produce an anti-apoptotic effect, as expression of the constitutively glycolytic PKM1 or treatment of WT cells with the PKIM2-stimulator TEPP-46 did not rescue cells from BimS-induced death. This argues that the anti-apoptotic effect induced by IB5 involves a nonglycolytic activity of PKM2. On the other hand, TEPP-46 significantly enhanced the cytoprotective activity of IB5, and a stably tetrameric mutant of PKM2, K422R, enhanced the effects of IB5 and TEPP-46. Taken together, these results argue that IB5's anti-apoptotic activity involves an active tetrameric conformation of PKM2.

In the absence of IB5, cells expressing PKM2 (K422R) for multiple passages displayed a degree of apoptosis resistance, and IB5 expression further enhanced this resistance. Such a cytoprotective effect of K422R may help explain why this mutation promoted oncogenesis in mice and occurred spontaneously in Bloom syndrome patient cells (Iqbal et al., 2014b). Bloom syndrome involves a mutation-prone mechanism and can therefore be considered an in vivo phenotypic selection process, in effect similar to the present inventors intrabody selection approach.

Finally, the anti-apoptotic activity induced by IB5 was not accompanied by changes in the levels of major Bcl-2-family proteins. In contrast, IB5 did upregulate Mfn1, and apoptosis resistance was ablated by Mfn1 deletion. This is consistent with reports that Mfn1 protein can directly oppose Bax-dependent apoptosis (Ryu et al., 2012). The present inventors' observation that mitochondria isolated from IB5-expressing cells were more resistant to Bax-mediated apoptosis (FIG. 6) may reflect increased levels of Mfn1 in mitochondria.

### Potential Implications

Without being limited to any theory, the present inventors discuss in the following section various potential implications and/or mechanism of action. The invention is not limited by any of these potential implications and/or mechanism of action but should be limited only with respect to the language set forth in the claims.

PKM2-deficient cells can form tumors in mice. Often the rapidly proliferating subset of tumor cells remodel glucose utilization by expressing low PKM1 levels, whereas nonproliferating tumor cells are more likely to express higher levels of PIKM1 (Israelsen et al., 2013). These observations reinforce the idea that reduced pyruvate kinase activity, and not necessarily PKM2 expression per se, is important for rapid cell proliferation. However, they also pose a question: if PKM2 is not strictly required for tumor formation, why is PKM2 expression overwhelmingly favored in human cancers? Although some human cancers harbor PKM2 loss-of-function mutations, these mutations are typically heterozygous. Thus, cancer cells presumably benefit from retaining at least one WT allele of the M2 isoform, which, unlike M1, provides adaptive glycolytic regulation and nonglycolytic functions (Iqbal et al., 2014a).

The present results suggest another potential benefit for cells expressing PKM2: resistance to apoptosis. Because PKM2 inhibits the central mechanism of apoptosis involving mitochondria, PKIVI2 could promote cell survival despite circumstances that would otherwise be cytotoxic. Without being limited to any theory, the present inventors conjecture that particular subsets of neoplastic or pre-neoplastic cells could engage this mechanism to survive under adverse conditions, favoring oncogenesis. Because glycolytically active PKM2 typically corresponds with lower rates of proliferation, without being limited to any theory, the present inventors hypothesize that this cell survival function of active PKM2 tetramers might be seen primarily in slowly proliferating tumor cell subsets.

Without being limited to any theory, it is proposed that IB5 most likely promotes cell survival by altering the interaction of PKM2 with one or more protein partners. It is tempting to hypothesize that IB5 mimics a natural PKM2-interacting protein. However, the identity of such a putative ligand is still unknown, as are the circumstances under which it is potentially engaged. Perhaps this cell survival function of PKM2 occurs only under specific conditions (e.g. allosteric activation of PKM2 combined with another regulatory event), which may explain why it has not been identified through conventional approaches. An anti-apoptotic function of PKM2 could be important both in cancer cells and in normal cell populations that preferentially express PKM2, such as macrophages (Barrero et al., 2013; Corcoran and O'Neill, 2016; Palsson-McDermott et al., 2015; Semba et al., 2016; Shirai et al., 2016) and podocytes in the kidney (Cheon et al., 2016; Qi et al., 2017).

It is believed that the herein described invention may be useful for treatment and/or prevention of a disease promoting apoptotic cell death in a subject. In one non-limiting embodiment, such disease can be selected from diabetes or diabetic nephropathy, non-alcoholic fatty liver disease (NALFD) or non-alcoholic steatohepatitis (NASH), and inflammatory dysfunction in coronary artery disease.

Other examples of implementations will become apparent to the reader in view of the teachings of the present description and as such, will not be further described here.

Note that titles or subtitles may be used throughout the present disclosure for convenience of a reader, but in no way should these limit the scope of the invention. Moreover, certain theories may be proposed and disclosed herein; however, in no way they, whether they are right or wrong, should limit the scope of the invention so long as the invention is practiced according to the present disclosure without regard for any particular theory or scheme of action.

All references cited throughout the specification are hereby incorporated by reference in their entirety for all purposes.

It will be understood by those of skill in the art that throughout the present specification, the term “a” used before a term encompasses embodiments containing one or more to what the term refers. It will also be understood by those of skill in the art that throughout the present specification, the term “comprising”, which is synonymous with “including,” “containing,” or “characterized by,” is inclusive or open-ended and does not exclude additional, un-recited elements or method steps.

Unless otherwise defined, all technical and scientific terms used herein have the same meaning as commonly understood by one of ordinary skill in the art to which this invention pertains. In the case of conflict, the present document, including definitions will control.

As used in the present disclosure, the terms “around”, “about” or “approximately” shall generally mean within the error margin generally accepted in the art. Hence, numerical quantities given herein generally include such error margin such that the terms “around”, “about” or “approximately” can be inferred if not expressly stated.

Although various embodiments of the disclosure have been described and illustrated, it will be apparent to those skilled in the art in light of the present description that numerous modifications and variations can be made. The scope of the invention is defined more particularly in the appended claims.

### REFERENCES

1. Anastasiou, D., Yu, Y., Israelsen, W. J., Jiang, J. K., Boxer, M. B., Hong, B. S., Tempel, W., Dimov, S., Shen, M., Jha, A., et al. (2012). Pyruvate kinase M2 activators promote tetramer formation and suppress tumorigenesis. Nature chemical biology 8, 839-847.
2. Barrero, C. A., Datta, P. K., Sen, S., Deshmane, S., Amini, S., Khalili, K., and Merali, S. (2013). HIV-1 Vpr modulates macrophage metabolic pathways: a SILAC-based quantitative analysis. PloS one 8, e68376.
3. Bender, T., and Martinou, J. C. (2013). Where killers meet—permeabilization of the outer mitochondrial membrane during apoptosis. Cold Spring Harbor perspectives in biology 5, a011106.
4. Bollenbach, T. J., Mesecar, A. D., and Nowak, T. (1999). Role of lysine 240 in the mechanism of yeast pyruvate kinase catalysis. Biochemistry 38, 9137-9145.
5. Boxer, M. B., Jiang, J. K., Vander Heiden, M. G., Shen, M., Skoumbourdis, A. P., Southall, N., Veith, H., Leister, W., Austin, C. P., Park, H. W., et al. (2010). Evaluation of substituted N,N′-diarylsulfonamides as activators of the tumor cell specific M2 isoform of pyruvate kinase. Journal of medicinal chemistry 53, 1048-1055.
6. Brown, J. M., and Attardi, L. D. (2005). The role of apoptosis in cancer development and treatment response. Nature reviews. Cancer 5, 231-237.
7. Brown, J. R., Tesar, B., Yu, L., Werner, L., Takebe, N., Mikler, E., Reynolds, H. M., Thompson, C., Fisher, D. C., Neuberg, D., et al. (2015). Obatoclax in combination with fludarabine and rituximab is well-tolerated and shows promising clinical activity in relapsed chronic lymphocytic leukemia. Leukemia & lymphoma 56, 3336-3342.
8. Cantor, J. R., and Sabatini, D. M. (2012). Cancer cell metabolism: one hallmark, many faces. Cancer discovery 2, 881-898.
9. Chen, L., Willis, S. N., Wei, A., Smith, B. J., Fletcher, J. I., Hinds, M. G., Colman, P. M., Day, C. L., Adams, J. M., and Huang, D. C. (2005). Differential targeting of prosurvival Bcl-2 proteins by their BH3-only ligands allows complementary apoptotic function. Mol Cell 17, 393-403.
10. Cheng, E. H., Wei, M. C., Weiler, S., Flavell, R. A., Mak, T. W., Lindsten, T., and Korsmeyer, S. J. (2001). BCL-2, BCL-X(L) sequester BH3 domain-only molecules preventing BAX- and BAK-mediated mitochondrial apoptosis. Mol Cell 8, 705-711.
11. Cheon, J. H., Kim, S. Y., Son, J. Y., Kang, Y. R., An, J. H., Kwon, J. H., Song, H. S., Moon, A., Lee, B. M., and Kim, H. S. (2016). Pyruvate Kinase M2: A Novel Biomarker for the Early Detection of Acute Kidney Injury. Toxicol Res 32, 47-56.
12. Chipuk, J. E., and Green, D. R. (2008). How do BCL-2 proteins induce mitochondrial outer membrane permeabilization? Trends in cell biology 18, 157-164.
13. Chipuk, J. E., Kuwana, T., Bouchier-Hayes, L., Droin, N. M., Newmeyer, D. D., Schuler, M., and Green, D. R. (2004). Direct activation of Bax by p⁵³ mediates mitochondrial membrane permeabilization and apoptosis. Science 303, 1010-1014.
14. Chipuk, J. E., Moldoveanu, T., Llambi, F., Parsons, M. J., and Green, D. R. (2010). The BCL-2 family reunion. Mol Cell 37, 299-310.
15. Choudhary, V., Kaddour-Djebbar, I., Alaisami, R., Kumar, M. V., and Bollag, W. B. (2014). Mitofusin 1 degradation is induced by a disruptor of mitochondrial calcium homeostasis, CGP37157: a role in apoptosis in prostate cancer cells. International journal of oncology 44, 1767-1773.
16. Christofk, H. R., Vander Heiden, M. G., Harris, M. H., Ramanathan, A., Gerszten, R. E., Wei, R., Fleming, M. D., Schreiber, S. L., and Cantley, L. C. (2008a). The M2 splice isoform of pyruvate kinase is important for cancer metabolism and tumour growth. Nature 452, 230-233.
17. Christofk, H. R., Vander Heiden, M. G., Wu, N., Asara, J. M., and Cantley, L. C. (2008b). Pyruvate kinase M2 is a phosphotyrosine-binding protein. Nature 452, 181-186.
18. Chu, B., Wang, J., Wang, Y., and Yang, G. (2015). Knockdown of PKM2 induces apoptosis and autophagy in human A549 alveolar adenocarcinoma cells. Molecular medicine reports 12, 4358-4363.
19. Corcoran, S. E., and O'Neill, L. A. (2016). HIFlalpha and metabolic reprogramming in inflammation. The Journal of clinical investigation 126, 3699-3707.
20. Croce, C. M., and Reed, J. C. (2016). Finally, An Apoptosis-Targeting Therapeutic for Cancer. Cancer research 76, 5914-5920.
21. Czabotar, P. E., Lessene, G., Strasser, A., and Adams, J. M. (2014). Control of apoptosis by the BCL-2 protein family: implications for physiology and therapy. Nat Rev Mol Cell Biol 15, 49-63.
22. Debrincat, M. A., Pleines, I., Lebois, M., Lane, R. M., Holmes, M. L., Corbin, J., Vandenberg, C. J., Alexander, W. S., Ng, A. P., Strasser, A., et al. (2015). BCL-2 is dispensable for thrombopoiesis and platelet survival. Cell death & disease 6, e1721.
23. Del Gaizo Moore, V., and Letai, A. (2013). BH3 profiling—measuring integrated function of the mitochondrial apoptotic pathway to predict cell fate decisions. Cancer letters 332, 202-205.
24. Dombrauckas, J. D., Santarsiero, B. D., and Mesecar, A. D. (2005). Structural basis for tumor pyruvate kinase M2 allosteric regulation and catalysis. Biochemistry 44, 9417-9429.
25. Dong, T., Yan, Y., Chai, H., Chen, S., Xiong, X., Sun, D., Yu, Y., Deng, L., and Cheng, F. (2015). Pyruvate kinase M2 affects liver cancer cell behavior through up-regulation of HIF-lalpha and Bcl-xL in culture. Biomedicine & pharmacotherapy=Biomedecine & pharmacotherapie 69, 277-284.
26. Du, H., Wolf, J., Schafer, B., Moldoveanu, T., Chipuk, J. E., and Kuwana, T. (2011). BH3 domains other than Bim and Bid can directly activate Bax/Bak. The Journal of biological chemistry 286, 491-501.
27. Elmore, S. (2007). Apoptosis: a review of programmed cell death. Toxicol Pathol 35, 495-516.
28. Franken, N. A., Rodermond, H. M., Stap, J., Haveman, J., and van Bree, C. (2006). Clonogenic assay of cells in vitro. Nature protocols 1, 2315-2319.
29. Fulda, S., and Debatin, K. M. (2007). HIF-1-regulated glucose metabolism: a key to apoptosis resistance? Cell cycle 6, 790-792.
30. Gandhi, L., Camidge, D. R., Ribeiro de Oliveira, M., Bonomi, P., Gandara, D., Khaira, D., Hann, C. L., McKeegan, E. M., Litvinovich, E., Hemken, P. M., et al. (2011). Phase I study of Navitoclax (ABT-263), a novel Bcl-2 family inhibitor, in patients with small-cell lung cancer and other solid tumors. Journal of clinical oncology: official journal of the American Society of Clinical Oncology 29, 909-916.
31. Gao, X., Wang, H., Yang, J. J., Liu, X., and Liu, Z. R. (2012). Pyruvate kinase M2 regulates gene transcription by acting as a protein kinase. Mol Cell 45, 598-609.
32. Gillies, L. A., Du, H., Peters, B., Knudson, C. M., Newmeyer, D. D., and Kuwana, T. (2015). Visual and functional demonstration of growing Bax-induced pores in mitochondrial outer membranes. Mol Biol Cell 26, 339-349.
33. Gillies, L. A., and Kuwana, T. (2014). Apoptosis regulation at the mitochondrial outer membrane. Journal of cellular biochemistry 115, 632-640.
34. Gines, A., Bystrup, S., Ruiz de Porras, V., Guardia, C., Musulen, E., MartinezCardus, A., Manzano, J. L., Layos, L., Abad, A., and Martinez-Balibrea, E. (2015). PKIM2 Subcellular Localization Is Involved in Oxaliplatin Resistance Acquisition in HT29 Human Colorectal Cancer Cell Lines. PloS one 10, e0123830.
35. Goldberg, M. S., and Sharp, P. A. (2012). Pyruvate kinase M2-specific siRNA induces apoptosis and tumor regression. The Journal of experimental medicine 209, 217-224.
36. Green, D. R. (2016). A BH3 Mimetic for Killing Cancer Cells. Cell 165, 1560.
37. Gui, D. Y., Lewis, C. A., and Vander Heiden, M. G. (2013). Allosteric regulation of PKM2 allows cellular adaptation to different physiological states. Science signaling 6, pe7.
38. Hilgendorf, K. I., Leshchiner, E. S., Nedelcu, S., Maynard, M. A., Calo, E., Ianari, A., Walensky, L. D., and Lees, J. A. (2013). The retinoblastoma protein induces apoptosis directly at the mitochondria. Genes & development 27, 1003-1015.
39. Hitosugi, T., Kang, S., Vander Heiden, M. G., Chung, T. W., Elf, S., Lythgoe, K., Dong, S., Lonial, S., Wang, X., Chen, G. Z., et al. (2009). Tyrosine phosphorylation inhibits PKM2 to promote the Warburg effect and tumor growth. Science signaling 2, ra73.
40. Hosios, A. M., Fiske, B. P., Gui, D. Y., and Vander Heiden, M. G. (2015). Lack of Evidence for PKM2 Protein Kinase Activity. Mol Cell 59, 850-857.
41. Hu, W., Lu, S. X., Li, M., Zhang, C., Liu, L. L., Fu, J., Jin, J. T., Luo, R. Z., Zhang, C. Z., and Yun, J. P. (2015). Pyruvate kinase M2 prevents apoptosis via modulating Bim stability and associates with poor outcome in hepatocellular carcinoma. Oncotarget 6, 6570-6583.
42. Iqbal, M. A., Gupta, V., Gopinath, P., Mazurek, S., and Bamezai, R. N. (2014a). Pyruvate kinase M2 and cancer: an updated assessment. FEBS letters 588, 2685-2692.
43. Iqbal, M. A., Siddiqui, F. A., Chaman, N., Gupta, V., Kumar, B., Gopinath, P., and Bamezai, R. N. (2014b). Missense mutations in pyruvate kinase M2 promote cancer metabolism, oxidative endurance, anchorage independence, and tumor growth in a dominant negative manner. The Journal of biological chemistry 289, 8098-8105.
44. Israelsen, W. J., Dayton, T. L., Davidson, S. M., Fiske, B. P., Hosios, A. M., Bellinger, G., Li, J., Yu, Y., Sasaki, M., Homer, J. W., et al. (2013). PKM2 isoform-specific deletion reveals a differential requirement for pyruvate kinase in tumor cells. Cell 155, 397-409.
45. Jiang, J. K., Boxer, M. B., Vander Heiden, M. G., Shen, M., Skoumbourdis, A. P., Southall, N., Veith, H., Leister, W., Austin, C. P., Park, H. W., et al. (2010). Evaluation of thieno[3,2-b]pyrrole[3,2-d]pyridazinones as activators of the tumor cell specific M2 isoform of pyruvate kinase. Bioorganic & medicinal chemistry letters 20, 3387-3393.
46. Jiang, Y., Li, X., Yang, W., Hawke, D. H., Zheng, Y., Xia, Y., Aldape, K., Wei, C., Guo, F., Chen, Y., et al. (2014a). PKM2 regulates chromosome segregation and mitosis progression of tumor cells. Mol Cell 53, 75-87.
47. Jiang, Y., Wang, Y., Wang, T., Hawke, D. H., Zheng, Y., Li, X., Zhou, Q., Majumder, S., Bi, E., Liu, D. X., et al. (2014b). PKM2 phosphorylates MLC2 and regulates cytokinesis of tumour cells. Nature communications 5, 5566.
48. Johnson-Farley, N., Veliz, J., Bhagavathi, S., and Bertino, J. R. (2015). ABT-199, a BH3 mimetic that specifically targets Bcl-2, enhances the antitumor activity of chemotherapy, bortezomib and JQ1 in “double hit” lymphoma cells. Leukemia & lymphoma, 1-7.
49. Juin, P., Geneste, O., Gautier, F., Depil, S., and Campone, M. (2013). Decoding and unlocking the BCL-2 dependency of cancer cells. Nature reviews. Cancer 13, 455-465.
50. Kim, D., Fiske, B. P., Birsoy, K., Freinkman, E., Kami, K., Possemato, R. L., Chudnovsky, Y., Pacold, M. E., Chen, W. W., Cantor, J. R., et al. (2015a). SHMT2 drives glioma cell survival in ischaemia but imposes a dependence on glycine clearance. Nature 520, 363-367.
51. Kim, D. J., Park, Y. S., Kang, M. G., You, Y. M., Jung, Y., Koo, H., Kim, J. A., Kim, M. J., Hong, S. M., Lee, K. B., et al. (2015b). Pyruvate kinase isoenzyme M2 is a therapeutic target of gemcitabine-resistant pancreatic cancer cells. Experimental cell research 336, 119-129.
52. Kipps, T. J., Eradat, H., Grosicki, S., Catalano, J., Cosolo, W., Dyagil, I. S., Yalamanchili, S., Chai, A., Sahasranaman, S., Punnoose, E., et al. (2015). A phase 2 study of the BH3 mimetic BCL2 inhibitor navitoclax (ABT-263) with or without rituximab, in previously untreated B-cell chronic lymphocytic leukemia. Leukemia & lymphoma, 1-8.
53. Kluck, R. M., Bossy-Wetzel, E., Green, D. R., and Newmeyer, D. D. (1997). The release of cytochrome c from mitochondria: a primary site for Bcl-2 regulation of apoptosis. Science 275, 1132-1136.
54. Kuwana, T., Bouchier-Hayes, L., Chipuk, J. E., Bonzon, C., Sullivan, B. A., Green, D. R., and Newmeyer, D. D. (2005a). BH3 domains of BH3-only proteins differentially regulate Bax-mediated mitochondrial membrane permeabilization both directly and indirectly. Mol Cell 17, 525-535.
55. Kuwana, T., Bouchier-Hayes, L., Chipuk, J. E., Bonzon, C., Sullivan, B. A., Green, D. R., and Newmeyer, D. D. (2005b). BH3 domains of BH3-only proteins differentially regulate Bax-mediated mitochondrial membrane permeabilization both directly and indirectly. Molecular Cell 17, 525-535.
56. Kuwana, T., Mackey, M. R., Perkins, G., Ellisman, M. H., Latterich, M., Schneiter, R., Green, D. R., and Newmeyer, D. D. (2002). Bid, bax, and lipids cooperate to form supramolecular openings in the outer mitochondrial membrane. Cell 111, 331-342.
57. Kuwana, T., Olson, N. H., Kiosses, W. B., Peters, B., and Newmeyer, D. D. (2016). Pro-apoptotic Bax molecules densely populate the edges of membrane pores. Scientific reports 6, 27299.
58. Kwon, O. H., Kang, T. W., Kim, J. H., Kim, M., Noh, S. M., Song, K. S., Yoo, H. S., Kim, W. H., Xie, Z., Pocalyko, D., et al. (2012). Pyruvate kinase M2 promotes the growth of gastric cancer cells via regulation of Bcl-xL expression at transcriptional level. Biochemical and biophysical research communications 423, 38-44.
59. Leverson, J. D., Zhang, H., Chen, J., Tahir, S. K., Phillips, D. C., Xue, J., Nimmer, P., Jin, S., Smith, M., Xiao, Y., et al. (2015). Potent and selective small-molecule MCL-1 inhibitors demonstrate on-target cancer cell killing activity as single agents and in combination with ABT-263 (navitoclax). Cell death & disease 6, e1590.
60. Li, C., Zhao, Z., Zhou, Z., and Liu, R. (2016). PKM2 Promotes Cell Survival and Invasion Under Metabolic Stress by Enhancing Warburg Effect in Pancreatic Ductal Adenocarcinoma. Digestive diseases and sciences 61, 767-773.
61. Li, M. X., and Dewson, G. (2015). Mitochondria and apoptosis: emerging concepts. F1000Prime Rep 7, 42.
62. Li, Z., Yang, P., and Li, Z. (2014). The multifaceted regulation and functions of PKM2 in tumor progression. Biochimica et biophysica acta 1846, 285-296.
63. Lieber, J., Armeanu-Ebinger, S., and Fuchs, J. (2015). The role of BH3-mimetic drugs in the treatment of pediatric hepatoblastoma. International journal of molecular sciences 16, 4190-4208.
64. Llambi, F., Moldoveanu, T., Tait, S. W., Bouchier-Hayes, L., Temirov, J., McCormick, L. L., Dillon, C. P., and Green, D. R. (2011). A unified model of mammalian BCL-2 protein family interactions at the mitochondria. Mol Cell 44, 517-531.
65. Lopez, J., and Tait, S. W. (2015). Mitochondrial apoptosis: killing cancer using the enemy within. British journal of cancer 112, 957-962.
66. Lunt, S. Y., Muralidhar, V., Hosios, A. M., Israelsen, W. J., Gui, D. Y., Newhouse, L., Ogrodzinski, M., Hecht, V., Xu, K., Acevedo, P. N., et al. (2015). Pyruvate kinase isoform expression alters nucleotide synthesis to impact cell proliferation. Mol Cell 57, 95-107.
67. Luo, W., Hu, H., Chang, R., Zhong, J., Knabel, M., O'Meally, R., Cole, R. N., Pandey, A., and Semenza, G. L. (2011). Pyruvate kinase M2 is a PHD3-stimulated coactivator for hypoxia-inducible factor 1. Cell 145, 732-744.
68. Luo, W., and Semenza, G. L. (2011). Pyruvate kinase M2 regulates glucose metabolism by functioning as a coactivator for hypoxia-inducible factor 1 in cancer cells. Oncotarget 2, 551-556.
69. Lv, L., Xu, Y. P., Zhao, D., Li, F. L., Wang, W., Sasaki, N., Jiang, Y., Zhou, X., Li, T. T., Guan, K. L., et al. (2013). Mitogenic and oncogenic stimulation of K433 acetylation promotes PKM2 protein kinase activity and nuclear localization. Mol Cell 52, 340-352.
70. Mazurek, S. (2011). Pyruvate kinase type M2: a key regulator of the metabolic budget system in tumor cells. The international journal of biochemistry & cell biology 43, 969-980.
71. Moley, K. H., and Mueckler, M. M. (2000). Glucose transport and apoptosis.

Apoptosis: an international journal on programmed cell death 5, 99-105.
72. Montero, J., Sarosiek, K. A., DeAngelo, J. D., Maertens, O., Ryan, J., Ercan, D., Piao, H., Horowitz, N. S., Berkowitz, R. S., Matulonis, U., et al. (2015). Drug-induced death signaling strategy rapidly predicts cancer response to chemotherapy. Cell 160, 977-989.
73. Morgan, H. P., O'Reilly, F. J., Wear, M. A., O'Neill, J. R., Fothergill-Gilmore, L. A., Hupp, T., and Walkinshaw, M. D. (2013). M2 pyruvate kinase provides a mechanism for nutrient sensing and regulation of cell proliferation. Proceedings of the National Academy of Sciences of the United States of America 110, 5881-5886.
74. Munoz-Pinedo, C., El Mjiyad, N., and Ricci, J. E. (2012). Cancer metabolism: current perspectives and future directions. Cell death & disease 3, e248.
75. Newmeyer, D. D., Farschon, D. M., and Reed, J. C. (1994). Cell-free apoptosis in Xenopus egg extracts: inhibition by Bcl-2 and requirement for an organelle fraction enriched in mitochondria. Cell 79, 353-364.
76. Newmeyer, D. D., and Ferguson-Miller, S. (2003). Mitochondria: releasing power for life and unleashing the machineries of death. Cell 112, 481-490.
77. Palsson-McDermott, E. M., Curtis, A. M., Goel, G., Lauterbach, M. A., Sheedy, F. J., Gleeson, L. E., van den Bosch, M. W., Quinn, S. R., Domingo-Fernandez, R., Johnston, D. G., et al. (2015). Pyruvate kinase M2 regulates Hif-lalpha activity and IL-ibeta induction and is a critical determinant of the warburg effect in LPS-activated macrophages. Cell metabolism 21, 65-80.
78. Palsson-McDermott, E. M., and O'Neill, L. A. (2013). The Warburg effect then and now: from cancer to inflammatory diseases. BioEssays: news and reviews in molecular, cellular and developmental biology 35, 965-973.
79. Parnell, K. M., Foulks, J. M., Nix, R. N., Clifford, A., Bullough, J., Luo, B., Senina, A., Vollmer, D., Liu, J., McCarthy, V., et al. (2013). Pharmacologic activation of PKM2 slows lung tumor xenograft growth. Molecular cancer therapeutics 12, 1453-1460.
80. Pyakurel, A., Savoia, C., Hess, D., and Scorrano, L. (2015). Extracellular regulated kinase phosphorylates mitofusin 1 to control mitochondrial morphology and apoptosis. Mol Cell 58, 244-254.
81. Qi, W., Keenan, H. A., Li, Q., Ishikado, A., Kannt, A., Sadowski, T., Yorek, M. A., Wu, I. H., Lockhart, S., Coppey, L. J., et al. (2017). Pyruvate kinase M2 activation may protect against the progression of diabetic glomerular pathology and mitochondrial dysfunction. Nature medicine.
82. Roberts, A. W., Advani, R. H., Kahl, B. S., Persky, D., Sweetenham, J. W., Carney, D. A., Yang, J., Busman, T. B., Enschede, S. H., Humerickhouse, R. A., et al. (2015). Phase 1 study of the safety, pharmacokinetics, and antitumour activity of the BCL2 inhibitor navitoclax in combination with rituximab in patients with relapsed or refractory CD20 lymphoid malignancies. British journal of haematology.
83. Ryu, S. W., Choi, K., Park, J. H., Park, Y. M., Kim, S., and Choi, C. (2012). Mitofusin 1 inhibits an apoptosis-associated amino-terminal conformational change in Bax, but not its mitochondrial translocation, in a GTPase-dependent manner. Cancer letters 323, 62-68.
84. Sarosiek, K. A., and Letai, A. (2016). Directly targeting the mitochondrial pathway of apoptosis for cancer therapy using BH3 mimetics—recent successes, current challenges and future promise. The FEBS journal 283, 3523-3533.
85. Schafer, B., Quispe, J., Choudhary, V., Chipuk, J. E., Ajero, T. G., Du, H., Schneiter, R., and Kuwana, T. (2009). Mitochondrial outer membrane proteins assist Bid in Bax-mediated lipidic pore formation. Mol Biol Cell 20, 2276-2285.
86. Semba, H., Takeda, N., Isagawa, T., Sugiura, Y., Honda, K., Wake, M., Miyazawa, H., Yamaguchi, Y., Miura, M., Jenkins, D. M., et al. (2016). HIF-lalpha-PDK1 axis-induced active glycolysis plays an essential role in macrophage migratory capacity. Nature communications 7, 11635.
87. Shi, H. S., Li, D., Zhang, J., Wang, Y. S., Yang, L., Zhang, H. L., Wang, X. H., Mu, B., Wang, W., Ma, Y., et al. (2010). Silencing of pkm2 increases the efficacy of docetaxel in human lung cancer xenografts in mice. Cancer Sci 101, 1447-1453.
88. Shirai, T., Nazarewicz, R. R., Wallis, B. B., Yanes, R. E., Watanabe, R., Hilhorst, M., Tian, L., Harrison, D. G., Giacomini, J. C., Assimes, T. L., et al. (2016). The glycolytic enzyme PKMV2 bridges metabolic and inflammatory dysfunction in coronary artery disease. The Journal of experimental medicine 213, 337-354.
89. Suryani, S., Carol, H., Chonghaile, T. N., Frismantas, V., Sarmah, C., High, L., Bornhauser, B., Cowley, M. J., Szymanska, B., Evans, K., et al. (2014). Cell and molecular determinants of in vivo efficacy of the BH3 mimetic ABT-263 against pediatric acute lymphoblastic leukemia xenografts. Clinical cancer research: an official journal of the American Association for Cancer Research 20, 4520-4531.
90. Swiecicki, P. L., Bellile, E., Sacco, A. G., Pearson, A. T., Taylor, J. M., Jackson, T. L., Chepeha, D. B., Spector, M. E., Shuman, A., Malloy, K., et al. (2016). A phase II trial of the BCL-2 homolog domain 3 mimetic AT-101 in combination with docetaxel for recurrent, locally advanced, or metastatic head and neck cancer. Investigational new drugs 34, 481-489.
91. Tuzlak, S., Kaufmann, T., and Villunger, A. (2016). Interrogating the relevance of mitochondrial apoptosis for vertebrate development and postnatal tissue homeostasis. Genes & development 30, 2133-2151.
92. Volkmann, N., Marassi, F. M., Newmeyer, D. D., and Hanein, D. (2014). The rheostat in the membrane: BCL-2 family proteins and apoptosis. Cell death and differentiation 21, 206-215.
93. Walensky, L. D., Pitter, K., Morash, J., Oh, K. J., Barbuto, S., Fisher, J., Smith, E., Verdine, G. L., and Korsmeyer, S. J. (2006). A stapled BID BH3 helix directly binds and activates BAX. Mol Cell 24, 199-210.
94. Wang, H. J., Hsieh, Y. J., Cheng, W. C., Lin, C. P., Lin, Y. S., Yang, S. F., Chen, C. C., Izumiya, Y., Yu, J. S., Kung, H. J., et al. (2014). JMJD5 regulates PKM2 nuclear translocation and reprograms HIF-lalpha-mediated glucose metabolism. Proceedings of the National Academy of Sciences of the United States of America 111, 279-284.
95. Wang, P., Sun, C., Zhu, T., and Xu, Y. (2015a). Structural insight into mechanisms for dynamic regulation of PKM2. Protein & cell 6, 275-287.
96. Wang, S., Ma, Y., Wang, P., Song, Z., Liu, B., Sun, X., Zhang, H., and Yu, J. (2015b). Knockdown of PKM2 Enhances Radiosensitivity of Non-small cell Lung Cancer. Cell biochemistry and biophysics 73, 21-26.
97. Waterhouse, N.J., Goldstein, J. C., Kluck, R. M., Newmeyer, D. D., and Green, D. R. (2001). The (Holey) study of mitochondria in apoptosis. Methods in cell biology 66, 365-391.
98. Wong, N., Ojo, D., Yan, J., and Tang, D. (2015). PKM2 contributes to cancer metabolism. Cancer letters 356, 184-191.
99. Wu, H., Yang, P., Hu, W., Wang, Y., Lu, Y., Zhang, L., Fan, Y., Xiao, H., and Li, Z. (2016). Overexpression of PKM2 promotes mitochondrial fusion through attenuated p⁵³ stability. Oncotarget.
100. Wu, S., and Le, H. (2013). Dual roles of PKM2 in cancer metabolism. Acta biochimica et biophysica Sinica 45, 27-35.
101. Xie, J., Yea, K., Zhang, H., Moldt, B., He, L., Zhu, J., and Lerner, R. A. (2014). Prevention of cell death by antibodies selected from intracellular combinatorial libraries. Chemistry & biology 21, 274-283.
102. Yamaguchi, R., Lartigue, L., Perkins, G., Scott, R. T., Dixit, A., Kushnareva, Y., Kuwana, T., Ellisman, M. H., and Newmeyer, D. D. (2008). Opal-mediated cristae opening is Bax/Bak and BH3 dependent, required for apoptosis, and independent of Bak oligomerization. Mol Cell 31, 557-569.
103. Yang, W., Xia, Y., Hawke, D., Li, X., Liang, J., Xing, D., Aldape, K., Hunter, T., Alfred Yung, W. K., and Lu, Z. (2012a). PKM2 phosphorylates histone H3 and promotes gene transcription and tumorigenesis. Cell 150, 685-696.
104. Yang, W., Xia, Y., Ji, H., Zheng, Y., Liang, J., Huang, W., Gao, X., Aldape, K., and Lu, Z. (2011). Nuclear PKM2 regulates beta-catenin transactivation upon EGFR activation. Nature 480, 118-122.
105. Yang, W., Zheng, Y., Xia, Y., Ji, H., Chen, X., Guo, F., Lyssiotis, C. A., Aldape, K., Cantley, L. C., and Lu, Z. (2012b). ERK1/2-dependent phosphorylation and nuclear translocation of PKM2 promotes the Warburg effect. Nature cell biology 14, 1295-1304.
106. Youle, R. J., and Strasser, A. (2008). The BCL-2 protein family: opposing activities that mediate cell death. Nat Rev Mol Cell Biol 9, 47-59.
107. Yuan, S., Qiao, T., Zhuang, X., Chen, W., Xing, N., and Zhang, Q. (2016). Knockdown of the M2 Isoform of Pyruvate Kinase (PKM2) with shRNA Enhances the Effect of Docetaxel in Human NSCLC Cell Lines In Vitro. Yonsei Med J 57, 1312-1323.
108. Zhang, H., Torkamani, A., Jones, T. M., Ruiz, D. I., Pons, J., and Lerner, R. A. (2011). Phenotype-information-phenotype cycle for deconvolution of combinatorial antibody libraries selected against complex systems. Proceedings of the National Academy of Sciences of the United States of America 108, 13456-13461.
109. Zhang, H., Wilson, I. A., and Lerner, R. A. (2012). Selection of antibodies that regulate phenotype from intracellular combinatorial antibody libraries. Proceedings of the National Academy of Sciences of the United States of America 109, 15728-15733.
110. Zhou, Z., Liu, Y., Qin, M., Sheng, W., Wang, X., Li, Z., and Zhong, R. (2014). Depletion of PKM2 leads to impaired glycolysis and cell death in 2-demethoxy-2,3-ethylenediamino hypocrellin B-photoinduced A549 cells. Journal of photochemistry and photobiology. B, Biology 134, 1-8.

1. A method for prevention or treatment of a disease promoting apoptotic cell death in a subject, comprising: contacting, in a cell of the subject, an antibody or fragment thereof with a pyruvate kinase M2 (PKM2) protein, the antibody or fragment thereof having binding specificity to PKM2.

2. The method of claim 1, wherein the disease is selected from diabetes or diabetic nephropathy, non-alcoholic fatty liver disease (NALFD) or non-alcoholic steatohepatitis (NASH), and inflammatory dysfunction in coronary artery disease.

3. The method of claim 1, further comprising administration to the subject of an effective amount of said antibody or fragment thereof.

4. The method of claim 3, wherein said antibody or fragment thereof is conjugated or coupled to a cell membrane permeable peptide.

5. The method of claim 4, wherein the cell membrane permeable peptide is the human immunodeficiency virus (HIV) trans-activator of transcription (Tat) peptide.

6. The method of claim 3, wherein said antibody or fragment thereof is in a composition comprising the antibody or fragment thereof and a membrane fusion liposome.

7. The method of claim 3, further comprising simultaneous or sequential administration of a PKM2-activating compound.

8. The method of claim 7, wherein the PKM2-activating compound includes thieno[3,2-b]pyrrole[3,2-d]pyridazinone NCGC00186528 (TEPP-46).

9. The method of claim 1, further comprising causing an intracellular expression of the antibody or fragment thereof in a cell expressing said PKM2.

10. The method of claim 9, wherein the causing an intracellular expression step includes administration of a gene delivery system to the subject, the gene delivery system including a nucleic acid molecule encoding the antibody or fragment thereof, wherein the system delivers the nucleic acid molecule into the cell expressing said PKM2.

11. The method of claim 10, wherein the system includes a non-viral system.

12. The method of 11, wherein the non-viral system includes a chitosan-based nanoparticle.

13. The method of 10, wherein the system includes a viral system.

14. The method of 13, wherein the viral system includes a DNA or RNA-based virus.

15. The method of claim 1, the antibody or fragment thereof having binding specificity to a PKM2 epitope comprising at least a portion of the amino acid sequence set forth in SEQ ID NO: 15.

16. The method of claim 15, wherein the PMK2 epitope includes an epitope formed by at least amino acids specific to the sequence set forth in SEQ ID NO: 15, wherein the specific characteristic is determined relative to the amino acid sequence set forth in SEQ ID NO: 16.

17. The method of claim 15, wherein the antibody or fragment thereof does not bind to a pyruvate kinase M1 (PKM1) epitope comprising at least a portion of the amino acid sequence set forth in SEQ ID NO: 16.

18. The method of claim 1, the antibody or fragment thereof having binding specificity to a PKIVI2 conformational epitope including amino acid residues contained in the amino acid sequence set forth in SEQ ID NO: 15.

19. The method of claim 1, wherein the antibody or fragment thereof includes the CDR1(H) set forth in SEQ ID NO: 3, the CDR2(H) set forth in SEQ ID NO: 4 and the CDR3(H) set forth in SEQ ID NO: 5; and the CDR1(L) set forth in SEQ ID NO: 6, the CDR2(L) set forth in SEQ ID NO: 7 and the CDR3(L) set forth in SEQ ID NO: 8.

20. The method of claim 1, wherein the antibody or fragment thereof includes the CDR1(H) set forth in SEQ ID NO: 9, the CDR2(H) set forth in SEQ ID NO: 10 and the CDR3(H) set forth in SEQ ID NO: 11; and the CDR1(L) set forth in SEQ ID NO: 12, the CDR2(L) set forth in SEQ ID NO: 13 and the CDR3(L) set forth in SEQ ID NO: 14

21. The method of 1, wherein the antibody or fragment thereof is a human, humanized, single chain (scFv) or chimeric antibody.

22. The method of claim 1, wherein the antibody or fragment thereof is a single chain (scFv) antibody having the amino acid sequence set forth in SEQ ID NO: 2.

23. The method of claim 1, wherein the antibody or fragment thereof is a single chain (scFv) antibody having the amino acid sequence set forth in SEQ ID NO: 1.

24. The method of claim 1, wherein the antibody or fragment thereof comprises
a. the CDR1(H) set forth in SEQ ID NO: 3, the CDR2(H) set forth in SEQ ID NO: 4 and the CDR3(H) set forth in SEQ ID NO: 5; or
b. the CDR1(H) set forth in SEQ ID NO: 9, the CDR2(H) set forth in SEQ ID NO: 10 and the CDR3(H) set forth in SEQ ID NO: 11,
wherein the CDR1(H), CDR2(H) and CDR3(H) are linked in tandem

25. A single chain (scFv) antibody comprising the amino acid sequence set forth in SEQ ID NO: 2.

26. A single chain (scFv) antibody comprising the amino acid sequence set forth in SEQ ID NO: 1.

27. An antibody having binding specificity to pyruvate kinase M2 (PKM2), the antibody comprising
a. the CDR1(H) set forth in SEQ ID NO: 3, the CDR2(H) set forth in SEQ ID NO: 4 and the CDR3(H) set forth in SEQ ID NO: 5; or
b. the CDR1(H) set forth in SEQ ID NO: 9, the CDR2(H) set forth in SEQ ID NO: 10 and the CDR3(H) set forth in SEQ ID NO: 11,
wherein the CDR1(H), CDR2(H) and CDR3(H) are linked in tandem.

28. A humanized, single chain (scFv) or chimeric antibody having binding specificity to a pyruvate kinase M2 (PKM2) epitope comprising at least a portion of the amino acid sequence set forth in SEQ ID NO: 15.

29. A humanized, single chain (scFv) or chimeric antibody of claim 28, the PKM2 epitope includes an epitope formed by at least amino acids specific to the sequence set forth in SEQ ID NO: 15, wherein the specific characteristic is determined relative to the amino acid sequence set forth in SEQ ID NO: 16.

30. A humanized, single chain (scFv) or chimeric antibody of claim 28, wherein the antibody or fragment thereof does not bind to a pyruvate kinase M1 (PKM1) epitope comprising at least a portion of the amino acid sequence set forth in SEQ ID NO: 16.

31. A humanized, single chain (scFv) or chimeric antibody, the antibody or fragment thereof having binding specificity to a PKM2 conformational epitope including amino acid residues contained in the amino acid sequence set forth in SEQ ID NO: 15.

32. A method for prevention or treatment of apoptotic cell death in a subject, the apoptotic cell death being associated with mitochondrial outer membrane permeabilization (MOMP), the method comprising: contacting, in a cell of the subject, an antibody or fragment thereof with a pyruvate kinase M2 (PKM2) protein, the antibody or fragment thereof having binding specificity to PKM2.

33. The method of claim 32, wherein the subject has a disease selected from diabetes or diabetic nephropathy, non-alcoholic fatty liver disease (NALFD) or non-alcoholic steatohepatitis (NASH), and inflammatory dysfunction in coronary artery disease.

34. The method of claim 32, further comprising administration to the subject of an effective amount of said antibody or fragment thereof.

35. The method of claim 34, wherein said antibody or fragment thereof is conjugated or coupled to a cell membrane permeable peptide.

36. The method of claim 35, wherein the cell membrane permeable peptide is the human immunodeficiency virus (HIV) trans-activator of transcription (Tat) peptide.

37. The method of claim 34, wherein said antibody or fragment thereof is in a composition comprising the antibody or fragment thereof and a membrane fusion liposome.

38. The method of claim 34, further comprising simultaneous or sequential administration of a PKM2-activating compound.

39. The method of claim 38, wherein the PKM2-activating compound includes thieno[3,2-b]pyrrole[3,2-d]pyridazinone NCGC00186528 (TEPP-46).

40. The method of claim 32, further comprising causing an intracellular expression of the antibody or fragment thereof in a cell expressing said PKM2.

41. The method of claim 40, wherein the causing an intracellular expression step includes administration of a gene delivery system to the subject, the gene delivery system including a nucleic acid molecule encoding the antibody or fragment thereof, wherein the system delivers the nucleic acid molecule into the cell expressing said PKM2.

42. The method of claim 41, wherein the system includes a non-viral system.

43. The method of 42, wherein the non-viral system includes a chitosan-based nanoparticle.

44. The method of 41, wherein the system includes a viral system.

45. The method of 44, wherein the viral system includes a DNA or RNA-based virus.

46. The method of claim 41, the antibody or fragment thereof having binding specificity to a PKM2 epitope comprising at least a portion of the amino acid sequence set forth in SEQ ID NO: 15.

47. The method of claim 46, wherein the PMK2 epitope includes an epitope formed by at least amino acids specific to the sequence set forth in SEQ ID NO: 15, wherein the specific characteristic is determined relative to the amino acid sequence set forth in SEQ ID NO: 16.

48. The method of claim 46, wherein the antibody or fragment thereof does not bind to a pyruvate kinase M1 (PKM1) epitope comprising at least a portion of the amino acid sequence set forth in SEQ ID NO: 16.

49. The method of claim 41, the antibody or fragment thereof having binding specificity to a PKIVI2 conformational epitope including amino acid residues contained in the amino acid sequence set forth in SEQ ID NO: 15.

50. The method of claim 41, wherein the antibody or fragment thereof includes the CDR1(H) set forth in SEQ ID NO: 3, the CDR2(H) set forth in SEQ ID NO: 4 and the CDR3(H) set forth in SEQ ID NO: 5; and the CDR1(L) set forth in SEQ ID NO: 6, the CDR2(L) set forth in SEQ ID NO: 7 and the CDR3(L) set forth in SEQ ID NO: 8.

51. The method of claim 41, wherein the antibody or fragment thereof includes the CDR1(H) set forth in SEQ ID NO: 9, the CDR2(H) set forth in SEQ ID NO: 10 and the CDR3(H) set forth in SEQ ID NO: 11; and the CDR1(L) set forth in SEQ ID NO: 12, the CDR2(L) set forth in SEQ ID NO: 13 and the CDR3(L) set forth in SEQ ID NO: 14.

52. The method of 41, wherein the antibody or fragment thereof is a human, humanized, single chain (scFv) or chimeric antibody.

53. The method of claim 41, wherein the antibody or fragment thereof is a single chain (scFv) antibody having the amino acid sequence set forth in SEQ ID NO: 2.

54. The method of claim 41, wherein the antibody or fragment thereof is a single chain (scFv) antibody having the amino acid sequence set forth in SEQ ID NO: 1.

55. The method of claim 41, wherein the antibody or fragment thereof comprises:
a. the CDR1(H) set forth in SEQ ID NO: 3, the CDR2(H) set forth in SEQ ID NO: 4 and the CDR3(H) set forth in SEQ ID NO: 5; or
b. the CDR1(H) set forth in SEQ ID NO: 9, the CDR2(H) set forth in SEQ ID NO: 10 and the CDR3(H) set forth in SEQ ID NO: 11
wherein the CDR1(H), CDR2(H) and CDR3(H) are linked in tandem.


SEQUENCE LISTING (16 sequences)

SEQ ID NO: 1 - protein, 269 aa, artificial
  Artificial intrabody clone 12
MAQVQLVQSGGGLVKPGGSLRLSCTASGFTFSTYWMHWFRQAPGKGLLWVSRINPDGSATIYADSVKGRFTISRDNAKNSLYLQMNSLRDEDTAVYYCARGHPLSGYPGYFDYWGQGTLVTVSSGGGGSGGGGSGVADPRLCSLSRLPSLHLLEHQQSHLHFTSGINVGAYRIYWYQQKPGSPPQFLLRYKSDSDKQQGSGVPSRFSGSRDASANAGILLISGLRSEDEADYYCAIWHSSAWVFGGGTKLTVLWGSGLASVDYKDDDDK

SEQ ID NO: 2 - protein, 265 aa, artificial
  artificial intrabody clone 5
MAQVQLVETGPGLVKPSETLSLRCTVSGGSFDNYYWNWIRQPPGKGLEYIGYVFPSTGATNYNPSLGSRVTISLDTSKNQFSLTLTSVTTADTAIYYCVRSGHDLWTGSTWFDPWGQWTTVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDIAVYYCQQRSNWPRTFGQGTKVEIKRGLGGLASVDYKDDDDK

SEQ ID NO: 3 - protein, 8 aa, artificial
  CDR1 (H) for IB5
GSFDNYYW

SEQ ID NO: 4 - protein, 8 aa, artificial
  CDR2(H)  for IB5
FPSTGATN

SEQ ID NO: 5 - protein, 10 aa, artificial
  CDR3(H) for IB5
HDLWTGSTWF

SEQ ID NO: 6 - protein, 10 aa, artificial
  CDR1(L) for IB5
SQSVSSSYLA

SEQ ID NO: 7 - protein, 6 aa, artificial
  CDR2(L) for IB5
ASSRAT

SEQ ID NO: 8 - protein, 8 aa, artificial
  CDR3(L) for IB5
QRSNWPRT

SEQ ID NO: 9 - protein, 8 aa, artificial
  CDR1(H) for IB12
FTFSTYWM

SEQ ID NO: 10 - protein, 8 aa, artificial
  CDR2(H) for IB12
NPDGSATI

SEQ ID NO: 11 - protein, 10 aa, artificial
  CDR3(H) for IB12
HPLSGYPGYF

SEQ ID NO: 12 - protein, 10 aa, artificial
  CDR1(L) for IB12
GINVGAYRIY

SEQ ID NO: 13 - protein, 6 aa, artificial
  CDR2(L) for IB12
SDSDKQ

SEQ ID NO: 14 - protein, 8 aa, artificial
  CDR3(L) for IB12
IWHSSAWV

SEQ ID NO: 15 - protein, 55 aa, homo sapiens
IAREAEAAIYHLQLFEELRRLAPITSDPTEATAVGAVEASFKCCSGAIIVLTKSG

SEQ ID NO: 16 - protein, 55 aa, homo sapiens
IAREAEAAMFHRKLFEELVRASSHSTDLMEAMAMGSVEASYKCLAAALIVLTESG